(19) United States
(12) Patent Application Publication
(10) Pub. No.: US 2026/0294816 A1
(43) Pub. Date: Oct. 1, 2026

(54) SLEEP FORMULATION

(71) Applicant: NightWise, LLC, Norcross, GA (US)

(72) Inventors: Daniel RINALDI, Duluth, GA (US); Scott FORSBERG, Morgan, UT (US)

(21) Appl. No.: 19/634,505

(22) Filed: Mar. 31, 2026

Related U.S. Application Data
(60) Provisional application No. 63/780,543, filed on Mar. 31, 2025.

(51) Int. Cl. A61K 9/48; A61K 31/197; A61K 31/198; A61K 31/4045; A61K 31/519; A61K 31/575; A61K 31/593; A61K 33/04; A61K 33/06; A61K 36/185; A61K 36/35; A61K 36/48; A61K 36/53; A61K 36/81; A61K 36/889; A61P 25/20

(52) U.S. Cl. CPC A61K 9/4808; A61K 9/4858; A61K 9/4866; A61K 31/197; A61K 31/198; A61K 31/4045; A61K 31/519; A61K 31/575; A61K 31/593; A61K 33/04; A61K 33/06; A61K 36/185; A61K 36/3486; A61K 36/35; A61K 36/48; A61K 36/53; A61K 36/81; A61K 36/889; A61P 25/20

(57) ABSTRACT

Described herein are delivery capsules and methods of making and using thereof. The delivery capsules can include a first population of lipid-based beads and a second population of lipid-based beads.

### CROSS-REFERENCE TO RELATED APPLICATION

This application claims priority to, and the benefit of U.S. Provisional Application 63/780,543, filed on Mar. 31, 2025, the contents of which is hereby incorporated by reference in its entirety.

### BACKGROUND

Sleep disorders such as insomnia or alterations in the sleep-wake cycle are common in human beings. It has been shown in a Gallup poll survey that 95% of the adult population had experienced insomnia (Rosekind, M., J Clin. Psychiatry, 1992, 53:4-6). The current literature maintains consistently that approximately one third of all people have sleep problems in any given year.

Sleep disorders can have serious consequences by themselves and can lead to numerous other medical problems. As a result, numerous remedies and methods have been created to deal with sleep disorders. See Treisman et al., “Insomnia,” Johns Hopkins POC-IT Center, 2010 pp. 1-3. Some examples of sleep medications include over-the-counter medicines such as TYLENOL PM™, compositions containing melatonin, herbal remedies, as well as pharmaceuticals such as zolpidem, quetiapine, and many others. While some of these sleep medications may be effective, tolerance generally develops for most medications requiring increasingly higher dosages. Other sleep medications either do not work well, serious side effects, or both. For example, some medications do not continue to work over extended periods and the patient wakes up during the night.

There is a significant need for a sleep composition and method that is safe and effective. There is also a need for a sleep composition that is effective over an extended period, and does not have significant side effects. The compositions and methods disclosed herein address these and other needs.

### SUMMARY

Provided herein are delivery capsules including a first population of lipid-based beads and a second population of lipid-based beads; wherein the first population of lipid-based beads encapsulates one or more sleep promoting active agents; and the second population of beads encapsulates one or more sleep quality active agents; wherein after administration of the delivery capsule the first population of lipid-based beads has an immediate burst release of the sleep promoting active agents, and the second population of lipid-based beads has a sustained release of the sleep quality active agents; and wherein each active agent is independently a a phytochemical, a herbal extract, a vitamin, a hormone, an amino acid, a mineral, enzymes, neurotransmitters, or combinations thereof.

Described herein are also methods for treating a sleep disorder or modifying or improving a sleep-wake cycle in a subject including, administering: a delivery capsule including a first population of lipid-based beads and a second population of lipid-based beads; wherein the first population of lipid-based beads encapsulates one or more sleep promoting active agents; and the second population of beads encapsulates one or more sleep quality active agents; wherein after administration of the delivery capsule the first population of lipid-based beads has an immediate burst release of the sleep promoting active agents, and the second population of lipid-based beads has a sustained release of the sleep quality active agents; and wherein each active agent is independently a phytochemical, a herbal extract, a vitamin, a hormone, an amino acid, a mineral, enzymes, neurotransmitters, or combinations thereof.

Described herein are also methods of reducing a symptom of menopause, the method including administering to a menopausal subject a delivery capsule including a first population of lipid-based beads and a second population of lipid-based beads; wherein the first population of lipid-based beads encapsulates one or more sleep promoting active agents and one or more menopause support active agents; and the second population of beads encapsulates one or more sleep quality active agents; wherein after administration of the delivery capsule the first population of lipid-based beads has an immediate burst release of the sleep promoting active agents and one or more menopause support active agents, and the second population of lipid-based beads has a sustained release of the sleep quality active agents; and wherein each active agent is independently a phytochemical, a herbal extract, a vitamin, a hormone, an amino acid, a mineral, enzymes, neurotransmitters, or combinations thereof.

Described herein are also methods for treating a sleep disorder or modifying or improving a sleep-wake cycle and reducing a symptom of menopause in a menopausal subject, the method including: administering to a menopausal subject a delivery capsule including a first population of lipid-based beads and a second population of lipid-based beads; wherein the first population of lipid-based beads encapsulates one or more sleep promoting active agents and one or more menopause support active agents; and the second population of beads encapsulates one or more sleep quality active agents; wherein after administration of the delivery capsule the first population of lipid-based beads has an immediate burst release of the sleep promoting active agents and one or more menopause support active agents, and the second population of lipid-based beads has a sustained release of the sleep quality active agents; and wherein each active agent is independently a phytochemical, a herbal extract, a vitamin, a hormone, an amino acid, a mineral, enzymes, neurotransmitters, or combinations thereof.

Described herein are also methods for reducing urinary frequency during sleep-wake cycle in a subject, the method including: administering to the subject a delivery capsule including a first population of lipid-based beads and a second population of lipid-based beads; wherein the first population of lipid-based beads encapsulates one or more sleep promoting active agents and one or more urinary support active agents; and the second population of beads encapsulates one or more sleep quality active agents; wherein after administration of the delivery capsule the first population of lipid-based beads has an immediate burst release of the sleep promoting active agents and one or more urinary support active agents, and the second population of lipid-based beads has a sustained release of the sleep quality active agents; and wherein each active agent is independently a phytochemical, a herbal extract, a vitamin, a hormone, an amino acid, a mineral, enzymes, neurotransmitters, or combinations thereof.

Described herein are also methods for reducing urinary frequency during sleep-wake cycle and treating a sleep disorder or modifying or improving the sleep-wake cycle in a subject, the method including: administering to the subject a delivery capsule including a first population of lipid-based beads and a second population of lipid-based beads; wherein the first population of lipid-based beads encapsulates one or more sleep promoting active agents and one or more urinary support active agents; and the second population of beads encapsulates one or more sleep quality active agents; wherein after administration of the delivery capsule the first population of lipid-based beads has an immediate burst release of the sleep promoting active agents and one or more urinary support active agents, and the second population of lipid-based beads has a sustained release of the sleep quality active agents; and wherein each active agent is independently a phytochemical, a herbal extract, a vitamin, a hormone, an amino acid, a mineral, enzymes, neurotransmitters, or combinations thereof.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1 is an image showing black beads and white bead.

FIG. 2 is an image of beads placed in “00” vegetarian capsule and introduced to heated water with pH adjustment and addition of digestive enzymes.

FIG. 3 is an image of left bottle containing the “white” beads after 30 minutes of agitation and heat. Right bottle containing the “dark” beads after the same procedure.

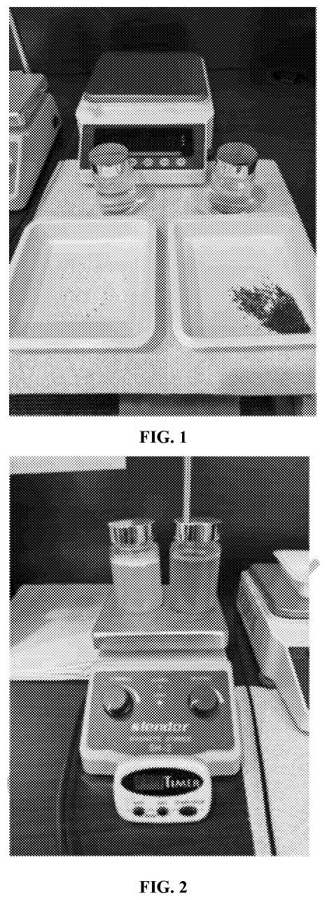

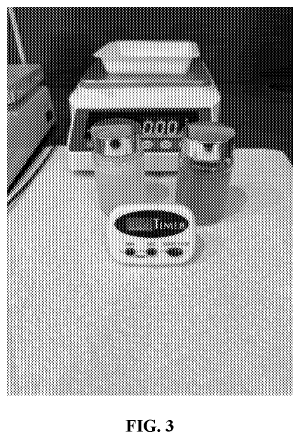

Like reference symbols in the various drawings indicate like elements.

### DETAILED DESCRIPTION

A number of embodiments of the disclosure have been described. Nevertheless, it will be understood that various modifications may be made without departing from the spirit and scope of the invention. Accordingly, other embodiments are within the scope of the following claims.

### Definitions

To facilitate understanding of the disclosure set forth herein, a number of terms are defined below. Unless defined otherwise, all technical and scientific terms used herein generally have the same meaning as commonly understood by one of ordinary skill in the art to which this disclosure belongs. Publications cited herein and the materials for which they are cited are specifically incorporated by reference.

### General Definitions

As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Accordingly, these terms are intended to not only cover the recited element(s) or step(s), but may also include other elements or steps not expressly recited. Furthermore, as used herein, the use of the terms “a”, “an”, and “the” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. A range may be construed to include the start and the end of the range. For example, a range of 10% to 20% (i.e., range of 10%-20%) can includes 10% and also includes 20%, and includes percentages in between 10% and 20%, unless explicitly stated otherwise herein.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inactivate”, “inactivating” and “inactivation” means to decrease or eliminate an activity, response, condition, disease, or other biological parameter due to a chemical (covalent bond formation) between the ligand and a its biological target.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage. In particular, the term “treatment” includes the alleviation, in part or in whole, of the symptoms of coronavirus infection (e.g., sore throat, blocked and/or runny nose, cough and/or elevated temperature associated with a common cold). Such treatment may include eradication, or slowing of population growth, of a microbial agent associated with inflammation.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms. As used herein, the term “preventing” a disorder or unwanted physiological event in a subject refers specifically to the prevention of the occurrence of symptoms and/or their underlying cause, wherein the subject may or may not exhibit heightened susceptibility to the disorder or event. In particular embodiments, “prevention” includes reduction in risk of coronavirus infection in patients. However, it will be appreciated that such prevention may not be absolute, i.e., it may not prevent all such patients developing a coronavirus infection, or may only partially prevent an infection in a single individual. As such, the terms “prevention” and “prophylaxis” may be used interchangeably.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount”. However, an appropriate “effective’ amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject. The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n-COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

A “control” is an alternative subject or sample used in an experiment for comparison purposes. A control can be “positive” or “negative.”

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician. Administration of the therapeutic agents can be carried out at dosages and for periods of time effective for treatment of a subject. In some embodiments, the subject is a human.

In the context of the present invention “menopause” includes peri-menopause, menopause and post-menopause, and in particular, symptoms that are caused or exacerbated by the decreased levels of estradiol (E₂) that attend peri-menopause, menopause and post-menopause. Exemplary menopausal symptoms include hot flashes, sweating secondary to vasomotor instability, psychological and emotional symptoms such as fatigue, irritability, insomnia, inability to concentrate, depression, memory loss, headache, anxiety, nervousness, intermittent dizziness, paresthesias, palpitations, tachycardia, nausea, constipation, diarrhea, arthralgia, myalgia, cold hands and feet, weight gain, urinary incontinence, vaginal dryness, loss of pelvic muscle tone, increased risk of cardiovascular disease and osteoporosis.

In the context of the present invention, “menopausal subject” and its verbal variants refers to an adult female, especially an adult female human, who has once attained menarche and who is experiencing peri-menopause, menopause or post-menopause. One of skill in the art of gynecology will be able to identify the diagnostic characteristics of the onset of menopause and identify a subject as being a “menopausal subject” by art-recognized clinical methods. A “symptom of menopause” is a symptom associated with one or more of peri-menopause, menopause or post-menopause. Symptoms of menopause include hot flashes and sweating secondary to vasomotor instability, fatigue, irritability, insomnia, inability to concentrate, depression, memory loss, headache, anxiety, nervousness, intermittent dizziness, paresthesias, palpitations, tachycardia, nausea, constipation, diarrhea, arthralgia, myalgia, cold hands and feet, weight gain, urinary incontinence, vaginal dryness and loss of pelvic muscle tone.

Reference will now be made in detail to specific aspects of the disclosed materials, compounds, compositions, articles, and methods, examples of which are illustrated in the accompanying Examples and Figures.

### Compositions

Described herein are delivery capsules including a first population of lipid-based beads and a second population of lipid-based beads. In some embodiments, the first population of lipid-based beads can encapsulate one or more sleep promoting active agents. In some embodiments, the second population of beads can encapsulate one or more sleep quality active agents. In some embodiments, after administration of the delivery capsule the first population of lipid-based beads can have an immediate burst release of the sleep promoting active agents. In some embodiments, the second population of lipid-based beads can have a sustained release of the sleep quality active agents. In some embodiments, each active agent can be independently a phytochemical, a herbal extract, a vitamin, a hormone, an amino acid, a mineral, enzymes, neurotransmitters, or combinations thereof.

In some embodiments, the first population of lipid-based beads, second population of lipid-based beads, or any combination thereof can further include one or more support active agent, one or more additional active agents, or any combination thereof.

In some embodiments, the first population of lipid-based beads can further include one or more support active agents. In some embodiments, the first population of lipid-based beads can further include one or more additional active agents. In some embodiments, the first population of lipid-based beads can further include one or more support active agents and one or more additional active agents.

In some embodiments, the support active agents can include, but are not limited to, one or more sleep support active agents, one or more menopause support active agents, one or more prostate support active agents, one or more urinary support active agents, pain relief active agent, restless leg relief active agent, gastrointestinal health active agent, hormonal support active agent (e.g., thyroid function), digestive support active agent, weight management active agent, immune support active agent, energy renewal active agent, physical recovery active agent, neurotransmitter recovery active agent, neuroprotection support active agent, stress relief active agent, mood balance active agent, skin and hair support active agent, jet lag relief active agent, muscle relaxation active agent, bone/joint support active agent, cognitive support active agent, respiratory support active agent, metabolic support (e.g., blood sugar balance) active agent, heart support active agent, or any combination thereof.

In some embodiments, the support active agents can include, but are not limited to, one or more sleep support active agents, one or more menopause support active agents, one or more prostate support active agents, one or more urinary support active agents, or any combination thereof.

In some embodiments, the first population of lipid-based beads can further include one or more menopause support active agents. In some embodiments, the first population of lipid-based beads can further include one or more urinary support active agents.

In some embodiments, the second population of lipid-based beads can further include one or more menopause support active agents. In some embodiments, the second population of lipid-based beads can further include one or more urinary support active agents.

In some embodiments, the first population of lipid-based beads and the second population of lipid-based beads can further include one or more menopause support active agents. In some embodiments, the first population of lipid-based beads and the second population of lipid-based beads can further include one or more urinary support active agents.

In some embodiments, the first population of lipid-based beads and the second population of lipid-based beads are present in the delivery capsule in a ratio of first population of lipid-based beads to second population of lipid-based beads of at least 1:1 (e.g., at least 1:2, at least 1:3, at least 1:4, at least 1:5, at least 1:6, at least 1:7, at least 1:8, or at least 1:9).

In some embodiments, the first population of lipid-based beads and the second population of lipid-based beads are present in the delivery capsule in a ratio of first population of lipid-based beads to second population of lipid-based beads of 1:10 or less (e.g., 1:9 or less, 1:8 or less, 1:7 or less, 1:6 or less, 1:5 or less, 1:4 or less, 1:3 or less, or 1:2 or less).

The first population of lipid-based beads and the second population of lipid-based beads are present in the delivery capsule in a ratio of first population of lipid-based beads to second population of lipid-based beads ranging from any of the minimum values described above to any of the maximum values described above. For example, in some embodiments, the first population of lipid-based beads and the second population of lipid-based beads are present in the delivery capsule in a ratio of first population of lipid-based beads to second population of lipid-based beads of from 1:1 to 1:10 (e.g., from 1:1 to 1:2, from 1:1 to 1:3, from 1:1 to 1:4, from 1:1 to 1:5, from 1:1 to 1:6, from 1:1 to 1:7, from 1:1 to 1:8, from 1:1 to 1:9, from 1:2 to 1:3, from 1:2 to 1:4, from 1:2 to 1:5, from 1:2 to 1:6, from 1:2 to 1:7, from 1:2 to 1:8, from 1:2 to 1:9, from 1:2 to 1:10, from 1:3 to 1:4, from 1:3 to 1:5, from 1:3 to 1:6, from 1:3 to 1:7, from 1:3 to 1:8, from 1:3 to 1:9, from 1:3 to 1:10, from 1:4 to 1:5, from 1:4 to 1:6, from 1:4 to 1:7, from 1:4 to 1:8, from 1:4 to 1:9, from 1:4 to 1:10, from 1:5 to 1:6, from 1:5 to 1:7, from 1:5 to 1:8, from 1:5 to 1:9, from 1:5 to 1:10, from 1:6 to 1:7, from 1:6 to 1:8, from 1:6 to 1:9, from 1:6 to 1:10, from 1:7 to 1:8, from 1:7 to 1:9, from 1:7 to 1:10, from 1:8 to 1:9, from 1:8 to 1:10, from 1:9 to 1:10).

In some embodiments, the second population of lipid-based beads includes a binder. In some embodiments, the first population of lipid-based beads can include wax.

In some embodiments, the wax and the binder can be present in the second population of lipid-based beads in a ratio of wax to binder of at least 5:1 (e.g., at least 10:1, at least 15:1, at least 20:1, at least 25:1, at least 30:1, at least 35:1, at least 40:1, at least 45:1, at least 50:1, at least 55:1, at least 60:1, at least 65:1, at least 70:1, at least 75:1, at least 80:1, at least 85:1, at least 90:1, at least 95:1, at least 100:1, at least 105:1, at least 110:1, at least 115:1, at least 120:1, at least 125:1, at least 130:1, at least 135:1, at least 140:1, or at least 145:1).

In some embodiments, the wax and the binder can be present in the second population of lipid-based beads in a ratio of wax to binder of 150:1 or less (e.g., 140:1 or less, 130:1 or less, 120:1 or less, 110:1 or less, 100:1 or less, 90:1 or less, 80:1 or less, 70:1 or less, 60:1 or less, 50:1 or less, 40:1 or less, 30:1 or less, 20:1 or less, or 10:1 or less)

The wax and the binder can be present in the second population of lipid-based beads in a ratio of wax to binder ranging from any of the minimum values described above to any of the maximum values described above. For example, in some embodiments, the wax and the binder can be present in the second population of lipid-based beads in a ratio of wax to binder of from 5:1 to 150:1 (e.g., from 5:1 to 125:1, from 5:1 to 100:1, from 5:1 to 50:1, from 5:1 to 25:1, from 5:1 to 15:1, from 10:1 to 150:1, from 10:1 to 125:1, from 10:1 to 100:1, from 10:1 to 50:1, from 10:1 to 25:1, from 25:1 to 150:1, from 25:1 to 125:1, from 25:1 to 100:1, from 25:1 to 75:1, from 25:1 to 50:1, from 50:1 to 150:1, from 50:1 to 125:1, from 50:1 to 100:1, from 50:1 to 75:1, from 75:1 to 150:1, from 75:1 to 125:1, from 75:1 to 100:1, from 100:1 to 150:1, or from 110:1 to 140:1). For example, in some embodiments, the wax and the binder can be present in the second population of lipid-based beads in a ratio of wax to binder 120:1.

In some embodiments, a delivery capsule, wherein capsule includes the components in proportions as set forth in Tables 1~4 of the examples.

### Active Agents

Examples of suitable sleep promoting active agents and/or sleep quality active agents can include, but are not limited to, y-aminobutyric acid (GABA), glycine, hydroxytryptophan or a derivative thereof, picamilon or a derivative thereof, L-taurine or a derivative thereof, nicotinamide or a derivative thereof, L-theanine or a derivative thereof, 4-amino-3-phenylbutyric acid or a derivative thereof, L-tryptophan or a derivative thereof, calcium, calcium D-glucarate or a derivative thereof, calcium gluconate or a derivative thereof, calcium lactate or a derivative thereof, zinc, iron, magnesium, magnesium glycinate or a derivative thereof, magnesium taurate or a derivative thereof, magnesium chloride, magnesium citrate, magnesium oxide, magnesium amino acid chelate, isoflavone or a derivative thereof, astaxanthin or a derivative thereof, phosphatidylserine or a derivative thereof, glutamine or a derivative thereof, phenibut 4-amino-3-phenylbutyric acid or a derivative thereof, a milk peptide or a derivative thereof, a milk protein hydrolysate or a derivative thereof, lactium, Kava, Skullcap, lemon balm extract, passion flower extract, hops extract, chamomile extract, ashwagandha extract, jujube extract, catnip extract, Ashwagandha extract, picamilon extract, extract from Magnolia officinalis (bark), extract from Holy Basil, Tulsi, extract from Humulus lupulus, extract from Phellodendron amurense (bark), extract from Valeriana officinalis (root), extract from Hemerocallis Julva var. sempervirens, extract from Albizzia julibrissin Durazz, eicosapentaenoic acid (EPA), extract from St. John's wort, extract from Bacopa monnieri, Chinese herbal medicines, extract from Apocynum venetum (luobuma), extract from Ganoderma lucidum (reishi), extract from Matricaria chamomilla, extract from Albizia julibrissin, extract from Melissa officinalis (leaf), extract from Hemerocallis Julva var. sempervirens, phosphatidyl serine, blends thereof, or combinations thereof. Extracts from Magnolia officinalis and Phellodendron amurense are commercially available, for example, under the trade name RELORA™.

In some embodiments, the sleep promoting active agent includes melatonin, L-theanine, γ-aminobutyric acid (GABA), magnesium, or any combination thereof. In some embodiments, the first population of lipid-based beads includes at least two sleep promoting active agents selected from the group consisting of melatonin, L-theanine, γ-aminobutyric acid (GABA), and magnesium.

In some embodiments, the one or more sleep promoting active agents are present in a concentration of at least 5% by weight (e.g., at least 10% by weight, at least 15% by weight, at least 20% by weight, at least 25% by weight, at least 30% by weight, at least 35% by weight, at least 40% by weight, at least 45% by weight, at least 50% by weight, at least 55% by weight, at least 60% by weight, at least 65% by weight, at least 70% by weight, or at least 75% by weight) based on a total weight of the first population of lipid-based beads.

In some embodiments, the one or more sleep promoting active agents are present in a concentration of 80% by weight or less (e.g., 10% by weight or less, 15% by weight or less, 20% by weight or less, 25% by weight or less, 30% by weight or less, 35% by weight or less, 40% by weight or less, 45% by weight or less, 50% by weight or less, 55% by weight or less, 60% by weight or less, 65% by weight or less, 70% by weight or less, or 75% by weight or less) based on a total weight of the first population of lipid-based beads.

The one or more sleep promoting active agents are present in a concentration ranging from any of the minimum values described above to any of the maximum values described above. For example, in some embodiments, the one or more sleep promoting active agents are present in a concentration of from 5% by weight to 80% by weight (e.g., from 5% by weight to 70% by weight, from 5% by weight to 60% by weight, from 5% by weight to 50% by weight, from 5% by weight to 40% by weight, from 5% by weight to 30% by weight, from 5% by weight to 20% by weight, from 5% by weight to 10% by weight, from 10% by weight to 80% by weight, from 10% by weight to 70% by weight, from 10% by weight to 60% by weight, from 10% by weight to 50% by weight, from 10% by weight to 40% by weight, from 10% by weight to 30% by weight, from 10% by weight to 20% by weight, from 20% by weight to 80% by weight, from 20% by weight to 70% by weight, from 20% by weight to 60% by weight, from 20% by weight to 50% by weight, from 20% by weight to 40% by weight, from 20% by weight to 30% by weight, from 30% by weight to 80% by weight, from 30% by weight to 70% by weight, from 30% by weight to 60% by weight, from 30% by weight to 50% by weight, from 30% by weight to 40% by weight, from 40% by weight to 80% by weight, from 40% by weight to 70% by weight, from 40% by weight to 60% by weight, from 40% by weight to 50% by weight, from 50% by weight to 80% by weight, from 50% by weight to 70% by weight, from 50% by weight to 60% by weight, from 60% by weight to 80% by weight, from 60% by weight to 70% by weight, or from 70% by weight to 80% by weight) based on a total weight of the first population of lipid-based beads.

In some embodiments, the sleep quality active agent includes hops extract, lemon balm extract, valerian root extract, melatonin, or any combination thereof.

In some embodiments, when present the melatonin is present in a concentration of at least 0.01% by weight (e.g., at least 0.05% by weight, at least 0.1% by weight, at least 0.5% by weight, at least 1% by weight, at least 2.5% by weight, at least 5% by weight, at least 7.5% by weight, at least 10% by weight, at least 12.5% by weight, at least 15% by weight, at least 20% by weight, or at least 25% by weight) based on a total weight of the first population of lipid-based beads.

In some embodiments, when present the melatonin is present in a concentration of 30% by weight or less (e.g., 25% by weight or less, 20% by weight or less, 15% by weight or less, 12.5% by weight or less, 10% by weight or less, 7.5% by weight or less, 5% by weight or less, 2.5% by weight or less, 1% by weight or less, 0.5% by weight or less, 0.25% by weight or less, 0.1% by weight or less, or 0.05% by weight or less) based on a total weight of the first population of lipid-based beads.

When present the melatonin is present in a concentration of ranging from any of the minimum values described above to any of the maximum values described above. For example, in some embodiments, when present the melatonin is present in a concentration of from 0.01% by weight to 30% by weight (e.g., from 0.01% by weight to 20% by weight, from 0.01% by weight to 10% by weight, from 0.01% by weight to 5% by weight, from 0.01% by weight to 2.5% by weight, from 0.01% by weight to 1% by weight, from 0.01% by weight to 0.5% by weight, from 0.01% by weight to 0.1% by weight, from 0.1% by weight to 30% weight, from 0.1% by weight to 20% by weight, from 0.1% by weight to 10% by weight, from 0.1% by weight to 5% by weight, from 0.1% by weight to 2.5% by weight, from 0.1% by weight to 1% by weight, from 0.1% by weight to 0.5% by weight, from 0.5% by weight to 30% weight, from 0.5% by weight to 20% by weight, from 0.5% by weight to 10% by weight, from 0.5% by weight to 5% by weight, from 0.5% by weight to 2.5% by weight, from 0.5% by weight to 1% by weight, 0.5% by weight to 1.5% by weight, from 1% by weight to 30% weight, from 1% by weight to 20% by weight, from 1% by weight to 10% by weight, from 1% by weight to 5% by weight, from 1% by weight to 2.5% by weight, from 2.5% by weight to 30% weight, from 2.5% by weight to 20% by weight, from 2.5% by weight to 10% by weight, from 2.5% by weight to 5% by weight, from 5% by weight to 30% weight, from 5% by weight to 20% by weight, from 5% by weight to 10% by weight, from 10% by weight to 30% weight, from 10% by weight to 20% by weight, or from 20% by weight to 30% weight) based on a total weight of the first population of lipid-based beads.

In some embodiments, when present the melatonin is present in a concentration of at least 0.01% by weight (e.g., at least 0.05% by weight, at least 0.1% by weight, at least 0.5% by weight, at least 1% by weight, at least 2.5% by weight, at least 5% by weight, at least 7.5% by weight, at least 10% by weight, at least 12.5% by weight, at least 15% by weight, at least 20% by weight, or at least 25% by weight) based on a total weight of the second population of lipid-based beads.

In some embodiments, when present the melatonin is present in a concentration of 30% by weight or less (e.g., 25% by weight or less, 20% by weight or less, 15% by weight or less, 12.5% by weight or less, 10% by weight or less, 7.5% by weight or less, 5% by weight or less, 2.5% by weight or less, 1% by weight or less, 0.5% by weight or less, 0.25% by weight or less, 0.1% by weight or less, or 0.05% by weight or less) based on a total weight of the second population of lipid-based beads.

When present the melatonin is present in a concentration of ranging from any of the minimum values described above to any of the maximum values described above. For example, in some embodiments, when present the melatonin is present in a concentration of from 0.01% by weight to 30% by weight (e.g., from 0.01% by weight to 20% by weight, from 0.01% by weight to 10% by weight, from 0.01% by weight to 5% by weight, from 0.01% by weight to 2.5% by weight, from 0.01% by weight to 1% by weight, from 0.01% by weight to 0.5% by weight, from 0.01% by weight to 0.1% by weight, from 0.1% by weight to 30% weight, from 0.1% by weight to 20% by weight, from 0.1% by weight to 10% by weight, from 0.1% by weight to 5% by weight, from 0.1% by weight to 2.5% by weight, from 0.1% by weight to 1% by weight, from 0.1% by weight to 0.5% by weight, from 0.5% by weight to 30% weight, from 0.5% by weight to 20% by weight, from 0.5% by weight to 10% by weight, from 0.5% by weight to 5% by weight, from 0.5% by weight to 2.5% by weight, from 0.5% by weight to 1% by weight, 0.5% by weight to 1.5% by weight, from 1% by weight to 30% weight, from 1% by weight to 20% by weight, from 1% by weight to 10% by weight, from 1% by weight to 5% by weight, from 1% by weight to 2.5% by weight, from 2.5% by weight to 30% weight, from 2.5% by weight to 20% by weight, from 2.5% by weight to 10% by weight, from 2.5% by weight to 5% by weight, from 5% by weight to 30% weight, from 5% by weight to 20% by weight, from 5% by weight to 10% by weight, from 10% by weight to 30% weight, from 10% by weight to 20% by weight, or from 20% by weight to 30% weight) based on a total weight of the second population of lipid-based beads.

In some embodiments, when present the melatonin is present in a concentration of at least 0.005% by weight (e.g., at least 0.05% by weight, at least 0.5% by weight, at least 1% by weight, at least 2% by weight, at least 3% by weight, or at least 4% by weight) based on a total weight of the delivery capsule.

In some embodiments, when present the melatonin is present in a concentration of 5% by weight or less (e.g., 4% by weight or less, 3% by weight or less, 2% by weight or less, 1% by weight or less, 0.5% by weight or less, or 0.05% by weight or less) based on a total weight of the delivery capsule.

When present the melatonin is present in a concentration of ranging from any of the minimum values described above to any of the maximum values described above. For example, in some embodiments, when present the melatonin is present in a concentration of from 0.005% by weight to 5% by weight (e.g., from 0.005% by weight to 4% by weight, from 0.005% by weight to 3% by weight, from 0.005% by weight to 2% by weight, from 0.005% by weight to 1% by weight, from 0.005% by weight to 0.5% by weight, from 0.005% by weight to 0.05% by weight, from 0.05% by weight to 4% by weight, from 0.05% by weight to 3% by weight, from 0.05% by weight to 2% by weight, from 0.05% by weight to 1% by weight, from 0.05% by weight to 0.5% by weight, from 0.5% by weight to 4% by weight, from 0.5% by weight to 3% by weight, from 0.5% by weight to 2% by weight, from 0.5% by weight to 1% by weight, from 1% by weight to 4% by weight, from 1% by weight to 3% by weight, from 1% by weight to 2% by weight, from 2% by weight to 4% by weight, from 2% by weight to 3% by weight, from 3% by weight to 4% by weight, from 3% by weight to 5% by weight, from 4% by weight to 5% by weight, from 2% by weight to 5% by weight, from 1% by weight to 5% by weight) based on a total weight of the delivery capsule.

In some embodiments, the second population of lipid-based beads includes at least two sleep quality active agents selected from the group consisting of hops extract, lemon balm extract, valerian root extract, and melatonin.

In some embodiments, the one or more sleep quality active agents are present in a concentration of at least 5% by weight (e.g., at least 10% by weight, at least 15% by weight, at least 20% by weight, at least 25% by weight, at least 30% by weight, at least 35% by weight, at least 40% by weight, at least 45% by weight, at least 50% by weight, at least 55% by weight, at least 60% by weight, at least 65% by weight, at least 70% by weight, or at least 75% by weight) based on a total weight of the second population of lipid-based beads.

In some embodiments, the one or more sleep quality active agents are present in a concentration of 80% by weight or less (e.g., 10% by weight or less, 15% by weight or less, 20% by weight or less, 25% by weight or less, 30% by weight or less, 35% by weight or less, 40% by weight or less, 45% by weight or less, 50% by weight or less, 55% by weight or less, 60% by weight or less, 65% by weight or less, 70% by weight or less, or 75% by weight or less) based on a total weight of the second population of lipid-based beads.

The one or more sleep quality active agents are present in a concentration ranging from any of the minimum values described above to any of the maximum values described above. For example, in some embodiments, the one or more sleep quality active agents are present in a concentration of from 5% by weight to 80% by weight (e.g., from 5% by weight to 70% by weight, from 5% by weight to 60% by weight, from 5% by weight to 50% by weight, from 5% by weight to 40% by weight, from 5% by weight to 30% by weight, from 5% by weight to 20% by weight, from 5% by weight to 10% by weight, from 10% by weight to 80% by weight, from 10% by weight to 70% by weight, from 10% by weight to 60% by weight, from 10% by weight to 50% by weight, from 10% by weight to 40% by weight, from 10% by weight to 30% by weight, from 10% by weight to 20% by weight, from 20% by weight to 80% by weight, from 20% by weight to 70% by weight, from 20% by weight to 60% by weight, from 20% by weight to 50% by weight, from 20% by weight to 40% by weight, from 20% by weight to 30% by weight, from 30% by weight to 80% by weight, from 30% by weight to 70% by weight, from 30% by weight to 60% by weight, from 30% by weight to 50% by weight, from 30% by weight to 40% by weight, from 40% by weight to 80% by weight, from 40% by weight to 70% by weight, from 40% by weight to 60% by weight, from 40% by weight to 50% by weight, from 50% by weight to 80% by weight, from 50% by weight to 70% by weight, from 50% by weight to 60% by weight, from 60% by weight to 80% by weight, from 60% by weight to 70% by weight, or from 70% by weight to 80% by weight) based on a total weight of the second population of lipid-based beads.

Examples of suitable support active agents can include, but are not limited to, red clover, ashwagandha extract, folic acid, vitamin B6, vitamin D, vitamin C, vitamin E, β-sitosterol, saw palmetto, nettle extract, vitamin D, selenium, tart cherry extract, 5-Hydroxytryptophan, L-Glycine, phosphatidylserine (PS) soy- or sunflower-derived, zinc, schisandra berry extract, magnolia bark extract, apigenin chamomile extract, L-Theanine, cinnamon extract, dihydroberberine (DHB), myo-inositol, chromium, iron, R-alpha-lipoic acid (Na-R-ALA), Gymnema sylvestre extract, banaba leaf extract, ginger root extract, Artichoke leaf extract, Akkermansia muciniphila, inulin, berberine HCl, ceylon cinnamon bark extract, curcumin, bitter melon extract, trans-resveratrol from Polygonum cuspidatum, fenugreek seed extract, licorice root extract, slippery elm bark, L-Glutamine, guggul extract, schisandra berry extract, lutein, Lion's mane extract, CDP-Choline, Bacopa monnieri extract, reishi mushroom extract, astragalus root extract, palmitoylethanolamide, stem bromelain, Boswellia serrata extract, collagen, dihydromyricetin (DHM) hovenia dulcis extract, milk thistle silymarin extract, benfotiamine, glucosamine sulfate, chondroitin sulfate, pterostilbenep, or any combination thereof.

Examples of suitable menopause support active agent can include, but are not limited to, red clover, ashwagandha extract, folic acid, vitamin D, or any combination thereof.

In some embodiments, the menopause support active agent can include red clover, ashwagandha extract, folic acid, and vitamin D. In some embodiments, the menopause support active agent can include red clover, ashwagandha extract, and folic acid.

In some embodiments, the first population of lipid-based beads can include a menopause support active agent including red clover, ashwagandha extract, folic acid, and vitamin D. In some embodiments, the first population of lipid-based beads can include a menopause support active agent including red clover, ashwagandha extract, and folic acid.

In some embodiments, the first population of lipid-based beads can include a menopause support active agent including red clover, ashwagandha extract, folic acid, and vitamin D; and a second population of lipid-based beads including a sleep quality active agent described herein. In some embodiments, the first population of lipid-based beads can include a menopause support active agent including red clover, ashwagandha extract, and folic acid; and a second population of lipid-based beads including a sleep quality active agent described herein.

In some embodiments, the first population of lipid-based beads can include a menopause support active agent including red clover, ashwagandha extract, folic acid, and vitamin D; and a second population of lipid-based beads including a sleep quality active agent described herein and a menopause support active agent described herein. In some embodiments, the first population of lipid-based beads can include a menopause support active agent including red clover, ashwagandha extract, and folic acid; and a second population of lipid-based beads including a sleep quality active agent described herein and a menopause support active agent described herein.

In some embodiments, the one or more support active agents are present in a concentration of from 10% by weight to 60% by weight (e.g., 20% by weight to 60% by weight) based on a total weight of the first population of lipid based beads.

In some embodiments, the one or more support active agents are present in a concentration of from 10% by weight to 60% by weight (e.g., 20% by weight to 60% by weight) based on a total weight of the second population of lipid based beads.

In some embodiments, the one or more support active agents are present in a concentration of from 15% by weight to 40% by weight (e.g., 20% by weight to 40% by weight) based on a total weight of the delivery capsule.

Examples of suitable urinary support active agent can include, but are not limited to, β-sitosterol, saw palmetto, nettle extract, vitamin D, selenium, or any combination thereof.

In some embodiments, the urinary support active agent can include β-sitosterol, saw palmetto, nettle extract, vitamin D, selenium, or any combination thereof. In some embodiments, the urinary support active agent can include β-sitosterol, saw palmetto, nettle extract, selenium, or any combination thereof.

In some embodiments, the first population of lipid-based beads can include urinary support active agent including β-sitosterol, saw palmetto, nettle extract, vitamin D, and selenium. In some embodiments, the first population of lipid-based beads can include urinary support active agent including β-sitosterol, saw palmetto, nettle extract, and selenium.

In some embodiments, the first population of lipid-based beads can include an urinary support active agent including β-sitosterol, saw palmetto, nettle extract, vitamin D, and selenium; and a second population of lipid-based beads including a sleep quality active agent described herein. In some embodiments, the first population of lipid-based beads can include an urinary support active agent including β-sitosterol, saw palmetto, nettle extract, and, selenium; and a second population of lipid-based beads including a sleep quality active agent described herein.

In some embodiments, the first population of lipid-based beads can include an urinary support active agent including β-sitosterol, saw palmetto, nettle extract, vitamin D, and selenium; and a second population of lipid-based beads including a sleep quality active agent described herein and an urinary support active agent described herein. In some embodiments, the first population of lipid-based beads can include a urinary support active agent including β-sitosterol, saw palmetto, nettle extract, and selenium; and a second population of lipid-based beads including a sleep quality active agent described herein and a urinary support active agent described herein.

Examples of suitable jet-lag support active agent can include, but are not limited to, Vitamin B6, Tart cherry extract, 5-Hydroxytryptophan, Ashwagandha root extract, Vitamin B6, L-Glycine, L-Tryptophan, Vitamin B12, Phosphatidylserine (PS) Soy- or sunflower-derived, Zinc, Schisandra berry extract, or any combination thereof.

In some embodiments, the jet-lag support active agent can include Vitamin B6, Tart cherry extract, 5-Hydroxytryptophan, Ashwagandha root extract, Vitamin B6, L-Glycine, or any combination thereof. In some embodiments, the jet-lag support active agent can include L-Tryptophan, Vitamin B12, Phosphatidylserine (PS) Soy- or sunflower-derived, Zinc, Schisandra berry extract, or any combination thereof.

In some embodiments, the first population of lipid-based beads can include a jet-lag support active agent including Vitamin B6, Tart cherry extract, 5-Hydroxytryptophan, Ashwagandha root extract, Vitamin B6, L-Glycine, or any combination thereof. In some embodiments, the second population of lipid-based beads can include a jet-lag support active agent including L-Tryptophan, Vitamin B12, Phosphatidylserine (PS) Soy- or sunflower-derived, Zinc, Schisandra berry extract, or any combination thereof.

In some embodiments, the first population of lipid-based beads can include a jet-lag support active agent including Vitamin B6, Tart cherry extract, 5-Hydroxytryptophan, Ashwagandha root extract, Vitamin B6, L-Glycine, or any combination thereof, and a second population of lipid-based beads including a sleep quality active agent described herein.

In some embodiments, the first population of lipid-based beads can include a jet-lag support active agent including Vitamin B6, Tart cherry extract, 5-Hydroxytryptophan, Ashwagandha root extract, Vitamin B6, L-Glycine, or any combination thereof; and a second population of lipid-based beads including a sleep quality active agent described herein and a jet-lag support active agent described herein. In some embodiments, the first population of lipid-based beads can include a jet-lag support active agent including Vitamin B6, Tart cherry extract, 5-Hydroxytryptophan, Ashwagandha root extract, Vitamin B6, L-Glycine, or any combination thereof; and a second population of lipid-based beads including a sleep quality active agent described herein and a jet-lag support active agent including L-Tryptophan, Vitamin B12, Phosphatidylserine (PS) Soy- or sunflower-derived, Zinc, Schisandra berry extract, or any combination thereof.

In some embodiments, the additional active agents can include, but are not limited to, hypnotics (e.g., zolpidem, zaleplon, triazolam, estazolam, temazepam, eszopiclone, ramelteon, and the like), antidepressants (e.g., amitriptyline, nortriptyline, tazodone, doxepin, isocarboxazid, amoxapine, impramine, tricyclic antidepressants, orexin antagonist (e.g., suvorexant, lemborexant, and the like); anxiolytics (benzodiazepines (e.g., alprazolam, clonazepam, diazepam, lorazepam, quazepam, flurazepam), buspirone, azapirones, beta-blockers (e.g., propranolol, atenolol, oxprenolol), SSRIS (selective serotonin reuptake inhibitors) like sertraline, fluoxetine, and escitalopram; and SNRIs (serotonin and norepinephrine reuptake inhibitors) like venlafaxine and duloxetine, and the like); sedatives (e.g., secobarbital, butabarbital, and the like); sleep disorder agents (e.g., hypnotics, antidepresents, anxiolytics, sedatives, antihistamines, melatonin receptor agonist (e.g., tasimelteon), daridorexant, and the like), antihistamines (e.g, diphenhydramine HCl, chlorpheniramine maleate, doxylamine, and the like); antipsychotic agents, neuroleptic agents, neuron blocking agents, antimuscarinic anticonvulsants (e.g., phenyloin sodium, pregabalin, gabapentin, and the like); antispasmodics such as belladonna alkaloids, dicyclomine hydrochloride, and the like; progestins include, but are not limited to, natural and synthetic compounds having progestational activity, such as, for example, progesterone, chlormadinone acetate, norethindrone, cyproterone acetate, norethindrone acetate, desogestrel, levonorgestrel, drospirenone, trimegestone, norgestrel, norgestimate, norelgestromin, etonogestrel, gestodene, and other natural and/or synthetic gestagens; estrogens including, but are not limited to, natural and synthetic compounds having estrogenic activity, such as, for example, estradiol (17β-estradiol), 17α-estradiol, estriol, estrone, and their esters, such as the acetate, sulfate, valerate or benzoate esters of these compounds, including, for example, estradiol 17β-cypionate, estradiol 17-propionate, estradiol 3-benzoate, and piperazine estrone sulfate; ethinyl estradiol; conjugated estrogens (natural and synthetic); mestranol; agonistic anti-estrogens; and selective estrogen receptor modulators; gonodotropin releasing hormone (GnRh) or anologs thereof such as deslorelin, avorelin, leuprolide, triptorelin, nafarelin, goserelin, buserelin, and fertirelin; fesolinetant, clonidine, ospemifene, androgens, or any combination thereof.

### Wax

In some embodiments, the first population of lipid-based beads can include wax.

Examples of suitable waxes can include but are not limited to, carnauba wax, beeswax, paraffin wax, glycerol monosterate, yellow wax, white beeswax, cetyl palmitate, rice bran wax, candelilla wax, hydrogenated vegetable oils, stearic acid, stearyl alcohol, microcrystalline cellulose wax blends, polyethylene glycol waxes, montan wax, or any combination thereof. In some embodiments, the wax can be glycerol monosterate.

In some embodiments, the wax can be present in a concentration of at least 20% by weight (e.g., at least 30% by weight, at least 40% by weight, at least 50% by weight, at least 60% by weight, or at least 70% by weight) based on a total weight of the first population of lipid based beads.

In some embodiments, the wax can be present in a concentration of 80% by weight or less (e.g., 70% by weight or less, 60% by weight or less, 50% by weight or less, 40% by weight or less, or 30% by weight or less) based on a total weight of the first population of lipid based beads.

The wax can be present in a concentration ranging from any of the minimum values described above to any of the maximum values described above. For example, in some embodiments, the wax can be present in a concentration of from 20% by weight to 80% by weight (e.g, from 20% to 70% by weight, from 20% to 60% by weight, from 20% to 50% by weight, from 20% to 40% by weight, from 20% to 30% by weight, from 30% to 80% by weight, from 30% to 70% by weight, from 30% to 60% by weight, from 30% to 50% by weight, from 30% to 40% by weight, from 40% to 80% by weight, from 40% to 70% by weight, from 40% to 60% by weight, from 40% to 50% by weight, from 50% to 80% by weight, from 50% to 70% by weight, from 50% to 60% by weight, from 60% to 80% by weight, from 60% to 70% by weight) based on a total weight of the first population of lipid based beads.

In some embodiments, the second population of lipid-based beads can include wax.

In some embodiments, the wax can be present in a concentration of at least 20% by weight (e.g., at least 30% by weight, at least 40% by weight, at least 50% by weight, at least 60% by weight, or at least 70% by weight) based on a total weight of the second population of lipid based beads.

In some embodiments, the wax can be present in a concentration of 80% by weight or less (e.g., 70% by weight or less, 60% by weight or less, 50% by weight or less, 40% by weight or less, or 30% by weight or less) based on a total weight of the second population of lipid based beads.

The wax can be present in a concentration ranging from any of the minimum values described above to any of the maximum values described above. For example, in some embodiments, the wax can be present in a concentration of from 20% by weight to 80% by weight (e.g, from 20% to 70% by weight, from 20% to 60% by weight, from 20% to 50% by weight, from 20% to 40% by weight, from 20% to 30% by weight, from 30% to 80% by weight, from 30% to 70% by weight, from 30% to 60% by weight, from 30% to 50% by weight, from 30% to 40% by weight, from 40% to 80% by weight, from 40% to 70% by weight, from 40% to 60% by weight, from 40% to 50% by weight, from 50% to 80% by weight, from 50% to 70% by weight, from 50% to 60% by weight, from 60% to 80% by weight, from 60% to 70% by weight) based on a total weight of the second population of lipid based beads.

### Binder

In some embodiments, the second population of lipid-based beads includes a binder. The binder can be biocompatible. “Biocompatible” or “biologically compatible”, as used herein, generally refer to binding agents that are, along with any metabolites or degradation products thereof, generally non-toxic to cells and tissues, and which do not cause any significant adverse. effects to cells and tissues when cells and tissues are incubated (e.g., cultured) in their presence. In some examples, the biocompatible binding agent can be generally recognized as safe (GRAS) compliant. GRAS status is an American Food and Drug Administration (FDA) designation that a chemical or active agent added to food is considered safe by experts, and so is exempted from the usual Federal Food, Drug, and Cosmetic Act (FFDCA) food additive tolerance requirements. In some aspects of the present disclosure, the binder can be a polymeric binding agent. Examples of suitable polymeric binding agents include, but are not limited to, methyl cellulose, ethyl cellulose, microcrystalline cellulose, croscarmellose sodium, dicalcium phosphate, cellulose, hypromellose, hydroxypropyl methylcellulose, carboxymethylcellulose, alkali metal carboxymethyl cellulose, carboxyethyl cellulose, hydroxymethyl cellulose, hydroxyethyl cellulose, hydroxypropyl cellulose, or combinations thereof. Other exemplary binders can include xanthan gum, viscarin, gelatin, starch, glucose, sucrose, polyvinyl pyrollidone, polyvinyl alcohol, gum tragacanth, gum karaya, sodium alginate, Laponite CP or SP, or magnesium aluminum silicate gel. The binder can also be coloring agents, taste masking agents, or combinations thereof. In some examples, cellulose-based binders are used.

In some embodiments, the binder can include methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC), hydroxyethylcarboxymethylcellulose (HECMC), carboxymethylhydroxyethylcellulose (CMHEC), or pharmaceutically acceptable salts thereof, or any combination thereof. In some embodiments, the second population of lipid-based beads includes hydroxypropyl methyl cellulose (HPMC).

In some embodiments, the binder can be present in a concentration of at least 0.1% by weight (e.g., at least 0.5% by weight, at least 1% by weight, at least 1.5% by weight, at least 2% by weight, at least 2.5% by weight, at least 5% by weight, at least 7.5% by weight, at least 10% by weight, at least 12.5% by weight, at least 15% by weight, at least 17.5% by weight) based on a total weight of the second population of lipid-based beads.

In some embodiments, the binder can be present in a concentration of 20% by weight or less (e.g., 15% by weight or less, 10% by weight or less, 5% by weight or less, 2.5% by weight or less, 1% by weight or less, or 0.5% by weight or less) based on a total weight of the second population of lipid-based beads.

The binder can be present in a concentration ranging from any of the minimum values described above to any of the maximum values described above. For example, in some embodiments, the binder can be present in a concentration of from 0.1% by weight to 20% by weight (e.g., from 0.1% by weight to 15% by weight, from 0.1% by weight to 10% by weight, from 0.1% by weight to 5% by weight, from 0.1% by weight to 2% by weight, from 0.1% by weight to 1% by weight, from 0.1% by weight to 0.5% by weight, from 0.5% by weight to 20%, from 0.5% by weight to 15% by weight, from 0.5% by weight to 10% by weight, from 0.5% by weight to 5% by weight, from 0.5% by weight to 2% by weight, from 0.5% by weight to 1% by weight, from 1% by weight to 20%, from 1% by weight to 15% by weight, from 1% by weight to 10% by weight, from 1% by weight to 5% by weight, from 1% by weight to 2% by weight, from 2% by weight to 20%, from 2% by weight to 15% by weight, from 2% by weight to 10% by weight, from 2% by weight to 5% by weight, from 5% by weight to 20%, from 5% by weight to 15% by weight, from 5% by weight to 10% by weight, from 10% by weight to 20%, from 10% by weight to 15% by weight, or from 15% by weight to 20%) based on a total weight of the second population of lipid-based beads.

In some embodiments, when the capsules are injected and digested, the timing of the release of the various active agents is in accordance with the timing of the bead's degradation in the gut or lower intestine.

In some embodiments, the active agents can be independently located in the core or throughout the beads.

In some embodiments, the beads can further include a coating layer to further change the rate of release of the active agent. Additional coating layers will generally result in a delayed and/or slower release of the active agent.

In some aspects, the coating layer can be formulated such that external factors such as pH and osmosis can result in a delayed and/or sustained release of the active agent.

The compositions can also include additional components to alter the consistency, appearance, taste, smell, or shelf life of the beads or individual ingredients in the bead. These components may be added as either an additional ingredient in the beads, as a coating layer on an ingredient, or as a coating layer on the beads. In some examples, a coloring agent is used to alter the color of the beads. In some embodiments, a taste masking agent is used to alter the taste of the beads.

### Modified Release Compositions

When administered to a subject, the disclosed compositions can release certain active agents at certain periods, rather than all at once. For example, the compositions can release the sleep promoting active agent immediately or within about 30 minutes of administration (immediate burst release). In some embodiments, the first population of lipid-based beads can have an immediate burst release of the sleep promoting active agent within about 30 minutes of administration of the capsule. For example, the first population of lipid-based beads can have an immediate burst release of the sleep promoting active agent from about 1 minute to about 30 minutes, about 1 minute to about 25 minutes, or about 1 minute to about 20 minutes after administration. In some examples, the first population of lipid-based beads can have an immediate burst release of the sleep promoting active agent within about 30 minutes, about 25 minutes, about 20 minutes, about 15 minutes, about 10 minutes, about 9 minutes, about 8 minutes, about 7 minutes, about 6 minutes, about 5 minutes, about 4 minutes, about 3 minutes, about 2 minutes, or about 1 minute of administration. All or most (for example, 80% or greater, or 90% or greater) of the sleep promoting active agent may be released within this time.

In some embodiments, at least 80% (e.g., at least 90%, at least 92.5%, at least 95%, at least 97.5%, or at least 99%) of the sleep promoting active agents are released from the first population of beads within 2 hours of administration of the capsule.

In some embodiments, the second population of lipid-based beads has a sustained release of the sleep promoting active agent from about 10 minutes to about 8 hours after administration of the capsule. In some embodiments, release the sleep quality active agent is over a period of about 6 hours of administration (sustained or burst release). In some aspects, the second population of lipid-based beads has a sustained release of the sleep promoting active agent within about 8 hours of administration. For example, the second population of lipid-based beads has a sustained release of the sleep promoting active agent from about 10 minutes to about 8 hours, about 10 minutes to about 7 hours, about 10 minutes to about 6 hours, about 2 hours to about 6 hours, or about 2.5 hours to about 4.5 hours, after administration. In some embodiments, the composition can provide the second population of lipid-based beads has a sustained release of the sleep promoting active agent within about 8 hours, about 7.5 hours, about 7 hours, about 6.5 hours, about 6 hours, about 5.5 hours, about 5 hours, or about 4.5 hours of administration. In some embodiments, the second population of lipid-based beads has a sustained release of the sleep promoting active agent after about 1 hour, about 1.5 hours, about 2 hours, about 2.5 hours, about 3 hours, about 3.5 hours, about 4 hours, or about 4.5 hours of administration. In some embodiments, delayed sustained release of at least a portion of the sleep quality active agents is within 2 to 5 hours of administration. In some embodiments, the sleep quality active agent has a delayed sustained release of 2.5-4.5 hours.

Substantially all (for example, 80% or greater, 90% or greater, 95% or greater, or 100%) of the sleep quality active agent may be released within this time.

In some embodiments, sustained release of the sleep quality active agents is selected from a delayed burst release, a delayed sustained release, and combinations thereof.

In some embodiments, release of the sleep quality active agents includes a delayed sustained release of the sleep quality active agents.

### Methods of Using

Described herein are also a methods for treating a sleep disorder or modifying or improving a sleep-wake cycle in a subject including, administering: a delivery capsule including a first population of lipid-based beads and a second population of lipid-based beads; wherein the first population of lipid-based beads encapsulates one or more sleep promoting active agents; and the second population of beads encapsulates one or more sleep quality active agents; wherein after administration of the delivery capsule the first population of lipid-based beads has an immediate burst release of the sleep promoting active agents, and the second population of lipid-based beads has a sustained release of the sleep promoting active agents; and wherein each active agent is independently a phytochemical, a herbal extract, a vitamin, a hormone, an amino acid, a mineral, or combinations thereof.

In some embodiments, the first population of lipid-based beads can further include one or more menopause support active agents, or one or more urinary support active agents. In some embodiments, the first population of lipid-based beads can further include one or more menopause support active agents. In some embodiments, the first population of lipid-based beads can further include one or more urinary support active agents.

In some embodiments, the subject can be a menopausal subject. In some embodiments, the menopausal subject can have at least one of the following symptoms: sweating secondary to vasomotor instability, night sweats, hot flashes fatigue, irritability, insomnia, inability to concentrate, depression, memory loss, headache, anxiety, nervousness, intermittent dizziness, paresthesias, palpitations and tachycardia as well as nausea, constipation, diarrhea, arthralgia, myalgia, cold hands and feet, weight gain, changes to the genitals, urinary incontinence, vaginal dryness, loss of pelvic muscle tone, increased risk of cardiovascular disease, or osteoporosis. In some embodiments, menopausal subject can have at least one of the following symptoms sweating secondary to vasomotor instability, night sweats, hot flashes, fatigue, irritability, insomnia, anxiety, or any combination thereof.

Described herein are also methods of reducing a symptom of menopause, the method including administering to a menopausal subject a delivery capsule including a first population of lipid-based beads and a second population of lipid-based beads; wherein the first population of lipid-based beads encapsulates one or more sleep promoting active agents and one or more menopause support active agents; and the second population of beads encapsulates one or more sleep quality active agents; wherein after administration of the delivery capsule the first population of lipid-based beads has an immediate burst release of the sleep promoting active agents and one or more menopause support active agents, and the second population of lipid-based beads has a sustained release of the sleep promoting active agents; and wherein each active agent is independently a phytochemical, a herbal extract, a vitamin, a hormone, an amino acid, a mineral, or combinations thereof.

Described herein are also methods for treating a sleep disorder or modifying or improving a sleep-wake cycle and reducing a symptom of menopause in a menopausal subject, the method including: administering to a menopausal subject a delivery capsule including a first population of lipid-based beads and a second population of lipid-based beads; wherein the first population of lipid-based beads encapsulates one or more sleep promoting active agents and one or more menopause support active agents; and the second population of beads encapsulates one or more sleep quality active agents; wherein after administration of the delivery capsule the first population of lipid-based beads has an immediate burst release of the sleep promoting active agents and one or more menopause support active agents, and the second population of lipid-based beads has a sustained release of the sleep promoting active agents; and wherein each active agent is independently a phytochemical, a herbal extract, a vitamin, a hormone, an amino acid, a mineral, or combinations thereof.

In some embodiments, the symptom of menopause can include, but are not limited to, sweating secondary to vasomotor instability, night sweats, hot flashes fatigue, irritability, insomnia, inability to concentrate, depression, memory loss, headache, anxiety, nervousness, intermittent dizziness, paresthesias, palpitations and tachycardia as well as nausea, constipation, diarrhea, arthralgia, myalgia, cold hands and feet, weight gain, changes to the genitals, urinary incontinence, vaginal dryness, loss of pelvic muscle tone, increased risk of cardiovascular disease, or osteoporosis. In some embodiments, the symptom of menopause can include sweating secondary to vasomotor instability, night sweats, hot flashes, fatigue, irritability, insomnia, anxiety, or any combination thereof.

Described herein are also methods for reducing urinary frequency during sleep-wake cycle in a subject, the method including: administering to the subject a delivery capsule including a first population of lipid-based beads and a second population of lipid-based beads; wherein the first population of lipid-based beads encapsulates one or more sleep promoting active agents and one or more urinary support active agents; and the second population of beads encapsulates one or more sleep quality active agents; wherein after administration of the delivery capsule the first population of lipid-based beads has an immediate burst release of the sleep promoting active agents and one or more urinary support active agents, and the second population of lipid-based beads has a sustained release of the sleep promoting active agents; and wherein each active agent is independently a phytochemical, a herbal extract, a vitamin, a hormone, an amino acid, a mineral, or combinations thereof. In some embodiments, the subject can suffer from incontinence or nocturia.

Described herein are also methods for reducing urinary frequency during sleep-wake cycle and treating a sleep disorder or modifying or improving the sleep-wake cycle in a subject, the method including: administering to the subject a delivery capsule including a first population of lipid-based beads and a second population of lipid-based beads; wherein the first population of lipid-based beads encapsulates one or more sleep promoting active agents and one or more urinary support active agents; and the second population of beads encapsulates one or more sleep quality active agents; wherein after administration of the delivery capsule the first population of lipid-based beads has an immediate burst release of the sleep promoting active agents and one or more urinary support active agents, and the second population of lipid-based beads has a sustained release of the sleep promoting active agents; and wherein each active agent is independently a phytochemical, a herbal extract, a vitamin, a hormone, an amino acid, a mineral, or combinations thereof.

In some embodiments, the methods can include administering a delivery capsule described herein.

In some embodiments, the subject can suffer from incontinence or nocturia.

Sleep disorder, as used herein includes, but is not limited to, insomnia, difficulty falling asleep, difficulty staying asleep, narcolepsy and its clinical manifestations such as sleep attacks, cataplexy, sleep paralysis, hypnagogic hallucinations, sleep apnea, hypersomnia, failure to wake-up feeling refreshed and rested or fatigue on waking, and related disorders. In some examples, the sleep disorder comprises fragmented sleep architecture. In some embodiments, the sleep disorder can include insomnia, narcolepsy, sleep apnea, hypersomnia, and combinations thereof.

In some aspects, treating a sleep disorder or modifying or improving the sleep-wake cycle in a subject includes improving sleep sensation determined by subjective judgment using psychological evaluation techniques or improving the sleep state determined objectively using techniques of estimating sleep and wakefulness states based on continuous recording of activity amounts. For example, the obstructive sleep apnea questionnaire can be used to examine the sleep sensation of the previous night to the present morning upon arising. The questionnaire is based on the five factors of sleepiness upon arising, sleep initiation and sleep maintenance, dream quality, recovery from fatigue, and elongation of sleep length and has been verified in regard to the reliability and validity of the respective factors.

Methods of sleep recovery including enhancing mental acuity or cognitive activity in a subject are also disclosed. Specifically, methods of sleep recovery after sleeping without unpleasant feeling or grogginess are disclosed. Sleep recovery can be determined objectively by measuring changes in the brain activity within the alpha frequency band using an electroencephalogram, or subjectively using the Toronto Hospital Alertness Test (THAT). In some embodiments, the active agents can be administered simultaneously, for example in the form of a single unit composition or separately.

This composition is not limited in particular in terms of administration method, number of times of administration, administration period, etc., and can be administered once or in a plurality of times by a suitable form of administration. In some embodiments, the composition is administered orally, once daily.

In some examples, the sleep disorder is dyssomnia. Dyssomnia can include psychophysiological insomnia, sleep state misperception, idiopathic insomnia, obstructive sleep apnea syndrome, central sleep apnea syndrome, central alveolar hypoventilation syndrome, periodic limb movement disorder, restless leg syndrome, inadequate sleep hygiene, environmental sleep disorder, altitude insomnia, adjustment sleep disorder, insufficient sleep syndrome, limit-setting sleep disorder, sleep-onset association disorder, nocturnal eating or drinking syndrome, hypnotic dependent sleep disorder, stimulant-dependent sleep disorder, alcohol-dependent sleep disorder, toxin-induced sleep disorder, time zone change (jet lag) syndrome, shift work sleep disorder, irregular sleep-wake pattern, delayed sleep phase syndrome, advanced sleep phase syndrome and non-24-hour sleep-wake disorder.

In some examples, the sleep disorder is a parasomnia. Parasomnia can include confusional arousals, sleepwalking and sleep terrors, rhythmic movement disorder, sleep starts, sleep talking and nocturnal leg cramps.

In some examples, the sleep disorder is associated with a medical or psychiatric disorder. The medical or psychiatric disorder can include psychoses, mood disorders, anxiety disorders, panic disorders, alcoholism, cerebral degenerative disorders, dementia, parkinsonism, fatal familial insomnia, sleep-related epilepsy, electrical status epilepticus of sleep, sleep-related headaches, sleeping sickness, nocturnal cardiac ischemia, chronic obstructive pulmonary disease, sleep-related asthma, sleep-related gastroesophageal reflux, peptic ulcer disease, fibrositis syndrome, osteoarthritis, rheumatoid arthritis, fibromyalgia and post-surgical sleep disorder.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are both chemically related may be substituted for the agents described herein while the same or similar results would be achieved. All such similar substitutes and modifications apparent to those skilled in the art are deemed to be within the spirit, scope and concept of the disclosure as defined by the appended claims.

By way of non-limiting illustration, examples of certain embodiments of the present disclosure are given below.

### EXAMPLES

### Example 1: Manufacturing Process of Modified Release Beads in Capsules

The process of manufacturing modified-release beads without using spray coating, granulation, or milling involves a series of steps to mix nutraceutical ingredients with waxes and binders. Here's a detailed explanation of the process:

### Ingredient Preparation and Mixing

### Dry Blending:

Ingredients: The active nutraceutical ingredients are combined with dry excipients such as binders and fillers. Common binders include microcrystalline cellulose, polyvinylpyrrolidone (PVP), or natural gums like acacia. Fillers such as lactose or dicalcium phosphate may also be included to ensure the desired bead size and consistency.

Mixing: The dry ingredients are thoroughly blended in a high-shear mixer or a tumbler mixer to achieve a uniform mixture. This step ensures even distribution of the active ingredient and excipients.

### Binder and Wax Addition:

A dry binder helps to form a cohesive mass. Microcrystalline cellulose or PVP can be used to provide the necessary binding properties. The addition of wax (e.g., carnauba wax, beeswax, or paraffin wax) serves as a matrix material that modulates the release of the active ingredient. The wax may be added in a solid form and melted during processing.

### Bead Formation

The blended mixture, including active ingredients, binders, and waxes, is compacted using a tablet press or roller compactor. This process forms a dense, uniform mass without the need for milling or granulation. Compaction ensures the ingredients are tightly bound together, facilitating the formation of beads.

### Shaping and Spheronization:

The compacted material is extruded through a die to form uniform strands. These strands are then cut into specific lengths. The cut segments are placed in a spheronizer, which rotates them at high speeds to form spherical beads. The spheronizer's action helps achieve a uniform size and shape for the beads.

### Cooling and Solidification

After the beads are formed, they are cooled to solidify the wax content. This step is crucial to stabilize the bead structure and lock the active ingredient within the matrix.

### Encapsulation

The solidified beads are filled into capsules, which can be made from gelatin or vegetarian alternatives like hydroxypropyl methylcellulose (HPMC). This is done using an automated capsule-filling machine that ensures uniform dosing and proper sealing.

### Ingredients

Active Ingredients: Nutraceutical compounds such as vitamins, minerals, amino acids, or herbal extracts.

Binders: Substances like microcrystalline cellulose, PVP, or natural gums that help in forming a cohesive mixture.

Waxes: Materials such as carnauba wax, beeswax, or paraffin wax that serve as the matrix for controlled release.

Fillers (Optional): Inert substances like lactose or dicalcium phosphate to adjust bead size and mass.

Machinery Required: high-shear mixer or tumbler mixer (for thorough blending of dry ingredients), tablet press or roller compactor (for compacting the blended mixture into a uniform mass), extruder and spheronizer (for forming and shaping the beads into uniform spheres), cooling Apparatus (to solidify the wax and stabilize the beads), capsule filling machine (for encapsulating the beads into capsules).

### Example 2: Preparation of Formulations

Capsules were prepared as detailed in Tables 1-6 and as described below.

TABLE 1
Exemplary Sleep Formulation 1
1-Capsule

| | Potency | Active Daily | Label | Total |
|---|---|---|---|---|
| | (% active) | Dose (mg) | Claiming | Weight (mg) |
| Sleep Latency Phase | | | | |
| (Immediate Release Beadlets) | | | | |
| Glycerol Monostearate | | 137.07 | 137.07 | 137.07 |
| L-theanine | 100.00% | 100.00 | 100.00 | 100.00 |
| GABA | 100.00% | 25.00 | 25.00 | 25.00 |
| Magnesium (as Oxide) | 59.4% | 5.00 | 5.00 | 8.42 |
| Melatonin | 98.5% | 3.0 | 3.00 | 3.05 |
| Total | | 270.07 | 270.07 | 273.54 |
| Sleep Maintenance Phase | | | | |
| (Delayed Release Beadlets) | | | | |
| Glycerol Monostearate | | 174.94 | 174.94 | 174.94 |
| Valerian Root extract | 100.0% | 90.00 | 90.00 | 90.00 |
| Hops Strobile PE | 100.0% | 60.00 | 60.00 | 60.00 |
| Lemon Balm Leaf | 100.0% | 20.00 | 20.00 | 20.00 |
| HPMC | | 2.50 | 2.50 | 2.50 |
| Melatonin | 98.5% | 2.00 | 2.00 | 2.03 |
| Total | | 349.44 | 349.44 | 349.47 |
| Net Fill Weight (1 capsule) | | 619.51 | 619.51 | 623.00 |

TABLE 2
Exemplary Sleep Formulation 2 (including menopause support
active agents) - 3 Stage controlled Release Melatonin

| | |
|---|---|
| Components | % Daily Value |

Instant Release Proprietary Blend:

| Red Cover (Trifolium pratense), L- | 401 | mg |
|---|---|---|
| Theanine, GABA(Gamma- | | |
| Aminobutyric Acid), Ashwagandha | | |
| extract, folic acid, vitamin D | | |
| Melatonin | 3 | |
| Glycerol Monostearate | 137 | mg |
| magnesium (magnesium oxide) | 2.5 | mg |

Controlled Release Proprietary Blend:

| Hops (Humulua Lupulus L.) | 80 | mg |
|---|---|---|
| (Strobiles), Lemon Balm (Melissa | | |
| Officinalis) (Leaf) | | |
| Melatonin | 2 | mg |
| Glycerol Monostearate | 175 | mg |
| magnesium (magnesium oxide) | 2.5 | mg |
| Hydroxypropyl Methylcellulose | 2.5 | mg |

(HPMC)

TABLE 3
Exemplary Sleep Formulation 3 (including urinary support
active agents) - 3 Stage controlled Release Melatonin

| | |
|---|---|
| Components | % Daily Value |

Instant Release Proprietary Blend:

| Beta-sitosterol, L-Theanine, | 296 | mg |
|---|---|---|
| GABA(Gamma-Aminobutyric Acid), | | |
| Saw Palmetto (Serenoa repens), Nettle | | |
| Extract (Urtica dioica), selenium (as | | |
| amino acid chelate), vitamin D | | |
| Melatonin | 3 | |
| Glycerol Monostearate | 137 | mg |
| magnesium (magnesium oxide) | 2.5 | mg |

Controlled Release Proprietary Blend:

| Controlled Release Proprietary Blend: | 80 | mg |
|---|---|---|
| Hops (Humulua Lupulus L.) | | |
| (Strobiles), Lemon Balm (Melissa | | |
| Officinalis) (Leaf) | | |
| Melatonin | 2 | mg |
| Glycerol Monostearate | 174 | mg |
| magnesium (magnesium oxide) | 2.5 | mg |
| Hydroxypropyl Methylcellulose | 2.5 | mg |

(HPMC)

TABLE 4
Exemplary Sleep Formulation 4 - 3
Stage controlled Release Melatonin

| | |
|---|---|
| Components | % Daily Value |

Instant Release Proprietary Blend:

| L-Theanine, GABA(Gamma- | 125 | mg |
|---|---|---|
| Aminobutyric Acid) | | |
| Melatonin | 3 | |
| Glycerol Monostearate | 137 | mg |
| magnesium (magnesium oxide) | 2.5 | mg |

Controlled Release Proprietary Blend:

| Controlled Release Proprietary Blend: | 80 | mg |
|---|---|---|
| Hops (Humulua Lupulus L.) | | |
| (Strobiles), Lemon Balm (Melissa | | |
| Officinalis) (Leaf) | | |
| Melatonin | 2 | mg |
| Glycerol Monostearate | 175 | mg |
| magnesium (magnesium oxide) | 2.5 | mg |
| Hydroxypropyl Methylcellulose | 2.5 | mg |

(HPMC)

TABLE 5
Exemplary Sleep Formulation 2 (including menopause support
active agents) - 3 Stage controlled Release Melatonin

| | |
|---|---|
| Components | % Daily Value |

Instant Release Proprietary Blend:

| Folate 748 mcg DFE (440 mcg Folic | 187% | |
|---|---|---|
| Acid) | | |
| Red Clover Extract (Trifolium | 254 | mg |
| pratense), L-Theanine, GABA | | |
| (Gamma-Aminobutyric Acid), | | |
| Ashwagandha extract, | | |
| Melatonin | 3 | |
| Glycerol Monostearate | 137 | mg |
| magnesium (magnesium oxide) | 2.5 | mg |

Controlled Release Proprietary Blend:

| Red Clover Extract (Trifolium | 209 | mg |
|---|---|---|
| Pratense); Hops (Humulua Lupulus L.) | | |
| (Strobiles), Lemon Balm (Melissa | | |
| Officinalis) (Leaf); Ashwagandha | | |
| Extract | | |
| Melatonin | 2 | mg |
| Glycerol Monostearate | 175 | mg |
| magnesium (magnesium oxide) | 2.5 | mg |
| Hydroxypropyl Methylcellulose | 2.5 | mg |

(HPMC)

Other Ingredients: Calcium Carbonate (for color)

TABLE 6
Exemplary Sleep Formulation 3 (including urinary support
active agents) - 3 Stage controlled Release Melatonin

| | |
|---|---|
| Components | % Daily Value |

Instant Release Proprietary Blend:

| Selenium (as amino acid chelate) 60 | 109% | |
|---|---|---|
| mcg | | |
| Beta-sitosterol, L-Theanine, GABA | 195 | mg |
| (Gamma-Aminobutyric Acid), Saw | | |
| Palmetto (Serenoa repens), Nettle | | |
| Extract (Urtica dioica), | | |
| Melatonin | 3 | |
| Glycerol Monostearate | 137 | mg |
| magnesium (magnesium oxide) | 2.5 | mg |

Controlled Release Proprietary Blend:

| Beta-sitosterol, Hops (Humulua | 150 | mg |
|---|---|---|
| Lupulus L.) (Strobiles), Lemon Balm | | |
| (Melissa Officinalis) (Leaf); Saw | | |
| Palmetto (Serenoa repens), Nettle | | |
| Extract (Urtica dioica), | | |
| Melatonin | 2 | mg |
| Glycerol Monostearate | 175 | mg |
| magnesium (magnesium oxide) | 2.5 | mg |
| Hydroxypropyl Methylcellulose | 2.5 | mg |

(HPMC)

Other Ingredients: calcium carbonate (for color)

### Example 3: Dissolution Evaluation

Approximately 30 mL of Deionized Water was inserted into two glass containers each and warmed to body temperature (~98 deg F.). The pH of the water was lowered to approximately 1.0-2.0 to mimic the physiological pH of human stomach acid, using Hydrochloric Acid. The addition of equal molar amounts of Pepsin and Lipase (20 mg of each) were also dissolved in the liquid medium.

Approximately 750 mg of “dark” beads were placed in the first container and 750 mg of “white” beads were introduced into the second container at time “0,” and the pH was measured again and found to be approximately 2.0-3.0.

The mixture was allowed to “incubate,” with continuous agitation, maintaining temperature (to mimic gastric churning/peristalsis for 30 and 90 minutes (of note: the average gastric emptying time in the human stomach is from 20-40 minutes, depending upon stomach contents, age, and sex).

At the end of the 30 minutes, the beads were carefully extracted from the aqueous-acid-enzyme solution and filtered. The beads were then allowed to dry to obtain accurate weights, indicating the dissolution of the beads during the digestion process. The findings are below.

### Results

“Dark” Bead (Delayed Release)

| Measurement | Time (0) | Time (30 mins) | Time (60 mins) |
|---|---|---|---|
| Weight | 750 mg | 650 mg | 516 mg |
| pH | 2.0 | 3.0 | N/A |
| Temp (F.) | 99.0 | 98.6 | N/A |
| Appearance | Clear | Cloudy/Darker Hue | Darker Hue |

“White” Bead (Immediate Release)

| Measurement | Time (0) | Time (30 mins) | Time (60 mins) |
|---|---|---|---|
| Weight | 750 mg | 230 mg | 146 mg |
| pH | 2.0 | 3.0 | N/A |
| Temp (F.) | 99.0 | 98.8 | N/A |
| Appearance | Clear | Cloudy and White | Cloudy and White |

The findings note an approximate 69.3% decrease in the overall mass of the “white” beads, compared to an 13.3% reduction in the overall mass of the “dark” beads at 30 minutes. Indicating, the majority (over 86%) of the “dark/delayed release” beads safely make passage into the small intestine, bypassing digestion by the harsh acidic and enzymatic environment of the stomach.

The “white/immediate release” beads experienced an approximate 69% reduction in overall mass, suggesting, a sizable amount of the beads is dissolved in the stomach, creating a “two-phase” release of active compounds (i.e. first release in the stomach, second release in the duodenum or small intestine).

Assuming a homogeneous mixture of actives and wax, 2.5 mg of Melatonin in the “white/immediate release” beads.

(2.5 mg of Melatonin/dose of white beads)×(57.3% reduction in mass)=Approximately 2.17 mg of Melatonin is released within 30 minutes. (1.5 mg of Melatonin/dose of dark beads)×(13.3% reduction in mass)=Approximately 0.19 mg of Melatonin is released within 30 minutes. Total Melatonin release from both beads at 30 minutes is approximately 2.37 mg.

### DISCUSSION

The human stomach is a dynamic organ utilized for food breakdown and to a lesser degree, digestion, and absorption of nutrients (the vast majority of nutrient absorption occurs in the small intestine. The pH of the average human stomach is from 1.5-3.0 (depending upon age, race, diet, medication, stomach contents and gender).

Once a material is ingested a chemical signal is sent from the stomach to the brain to release Hydrogen Ions from small transporters called “proton pumps.” These hydrogen ions rapidly form hydrochloric acid and dramatically decrease the stomach lumen pH to aid in digestion. A small amount of stomach pepsin, used to break down proteins and stomach lipase, used to break down fats is also released. The action of “churning” or gastric peristalsis also begins and further assists in breaking down food.

To mitigate the breakdown of certain ingested substances prior to entering the small intestine, “coatings” of various materials have been used to envelop active materials allowing for passage into the small intestine.

Once inside the first part of the small intestine (duodenum) the pH rises dramatically to around 6-7.5. Where pancreatic enzymes and other biliary products are released to further breakdown partially digested food into absorbable constituents.

### CONCLUSIONS

Total Melatonin release from both beads within 30 minutes is approximately 2.37 mg. Although, this is a simulated in-vitro experiment to test that the coating on both beads is engineered to dissolve and “release” it's active ingredients at certain points throughout the digestion and absorption process, in-vivo or human clinical studies are warranted to validate these findings.

### Example 4: Sample Formulations

### Circadian Rhythm Synchronization

Melatonin secretion, core body temperature drop, and suprachiasmatic nucleus (SCN) pacemaker entrainment are all phase-dependent processes initiating in early evening and completing through mid-sleep. The suprachiasmatic nucleus governs circadian timing and responds to both light cues and endogenous oscillator feedback.

Immediate release delivers melatonin-supporting ingredients at ingestion to support sleep-onset entrainment. The control layer holds additional release for approximately 1.5 to 2 hours, allowing the initial circadian cue to take effect. Delayed release then sustains rhythm stability through the mid-sleep window, paralleling the extended SCN entrainment arc.

Composition for Sleep and Jet Lag (For Jet Lag, Night Shifts, and Disrupted Sleep Schedules). This is the first sleep supplement built specifically for the estimated 35 million Americans who regularly cross time zones business travelers, flight crews, military personnel, and frequent flyers as well as night shift workers and those living with disrupted sleep schedules. What they experience isn't just fatigue. It's a full disruption of the body's internal clock marked by daytime exhaustion, nighttime wakefulness, and a loss of rhythm that a single-dose melatonin tablet simply cannot reset.

The Immediate Release phase delivers the circadian reset signal and calms the nervous system at the new local bedtime, the Control Layer then holds all further release for approximately 1.5 to 2 hours allowing the body to begin anchoring to the new time zone without interruption before the Delayed Release phase activates precisely timed to the window when jet-lagged travelers most commonly wake and cannot return to sleep, delivering a second phase of circadian and sleep support that extends and consolidates sleep through the critical early morning hours in the new time zone.

TABLE 7
Sleep and Jet Lag Formulation

| Components | amount |
|---|---|
| Immediate Release | |
| GABA | 10 mg-200 mg |
| L-Theanine | 20 mg-200 mg |
| Magnesium bisglycinate | 5 mg-250 mg |
| Melatonin | 0.5 mg-5 mg |
| Vitamin B6 | 3 mg-55 mg |
| Tart cherry extract | 120 mg-600 mg |
| 5-Hydroxytryptophan | 25 mg-125 mg |
| Ashwagandha root extract | 75 mg-375 mg |
| Vitamin B6 | 1.25 mg-12.5 mg |
| L-Glycine | 750 mg-3,750 mg |
| Delayed Release | |
| Magnesium bisglycinate | 5 mg-250 mg |
| Melatonin | 0.5 mg-5 mg |
| Hops | 10 mg-100 mg |
| Lemon balm | 15 mg-125 mg |
| L-Tryptophan | 63 mg-625 mg |
| Vitamin B12 | 250 mcg-1,875 mcg |
| Phosphatidylserine (PS) Soy- or | 25 mg-125 mg |
| sunflower-derived | |
| Zinc bisglycinate or picolinate | 2.5 mg-18.75 mg |
| Schisandra berry extract | 50 mg-500 mg |

### Cortisol Rhythm Modulation

Cortisol follows a precise 24-hour rhythm, reaching its lowest point near midnight and rising sharply in the pre-dawn hours as part of the Cortisol Awakening Response. Dysregulation of this rhythm produces nighttime hyperarousal, mid-sleep awakenings in the 2 to 4 AM window, and stress-driven insomnia. The HPA axis is the governing system.

Immediate release delivers cortisol-buffering and sleep-stabilizing ingredients at ingestion to address early-night hyperarousal. The Control layer preserves the quiescent midnight window without additional input. Delayed Release targets the 2 to 4 AM period when pre-dawn cortisol elevation begins, addressing the primary mechanism behind middle-of-the-night awakenings.

Many insomnia presentations are cortisol timing problems rather than melatonin deficiencies.

Composition for Sleep and Stress Response (Quiets the Racing Mind That Wakes You at 3 AM). This is the first sleep supplement built specifically for the estimated 33 million Americans who fall asleep without difficulty and then wake between 2 and 4 AM with a racing mind, a surge of anxiety, and a cortisol spike that makes returning to sleep feel impossible a pattern that is not insomnia in the traditional sense but a specific and identifiable failure of the body's overnight cortisol regulation that no single-release sleep aid is designed to address.

The immediate release phase calms the nervous system and begins quieting the cortisol and stress hormone pathways at bedtime before they can build to the level that triggers the 3 AM waking, the control layer then holds all further release for approximately 1.5 to 2 hours allowing the body to move undisturbed through the early deep sleep cycles before the delayed release phase activates precisely timed to the 2 to 4 AM window when cortisol begins its earliest overnight rise, the stress response is most likely to fire, and the racing thoughts that pull people out of sleep are at their most disruptive.

TABLE 8
Sleep and stress response Formulation

| Components | amount |
|---|---|
| Immediate Release | |
| GABA | 10 mg-200 mg |
| L-Theanine | 20 mg-200 mg |
| Magnesium bisglycinate | 5 mg-250 mg |
| Melatonin | 0.5 mg-5 mg |
| Ashwagandha root extract | 75 mg-375 mg |
| Phosphatidylserine | 25 mg-125 mg |
| Magnolia bark extract | 50 mg-250 mg |
| Apigenin | 12.5 mg-62.5 mg |
| Delayed Release | |
| Magnesium bisglycinate | 5 mg-250 mg |
| Melatonin | 0.5 mg-5 mg |
| Hops | 10 mg-100 mg |
| Lemon balm | 15 mg-125 mg |
| L-Theanine: | 20 mg-200 mg |
| Phosphatidylserine: | 25 mg-125 mg |
| Ashwagandha root extract: | 37.5 mg-187.5 mg |
| Apigenin chamomile extract: | 12.5 mg-62.5 mg |

### Metabolic and Digestive Rhythm Support

Insulin sensitivity, glucose regulation, and digestive motility all follow circadian patterns. Nighttime glucose dysregulation and disrupted GI motility are increasingly recognized as drivers of sleep fragmentation. Leptin, ghrelin, and GLP-1 receptor dynamics are circadian-regulated and interact with sleep architecture.

Immediate release supports initial glycemic stability and GI comfort at ingestion. The Control layer allows metabolic settling in the early sleep period. Delayed release delivers metabolically active ingredients timed to the overnight recovery window, approximately 1.5 to 2 hours after ingestion, when hepatic glucose production and GI motility are at their nocturnal nadir.

Composition for Sleep and Metabolic Support (For blood sugar that won't stay stable overnight, digestion that needs to reset, and the sleep that metabolic imbalance keeps interrupting). This is the first sleep supplement built specifically for the estimated 88 million American adults with prediabetes or metabolic imbalance plus the tens of millions more managing blood sugar, weight, or digestive health who have discovered that unstable overnight glucose, the cortisol spikes that follow blood sugar crashes, and the digestive discomfort that peaks in the middle of the night are quietly destroying the sleep architecture that metabolic health depends on to repair itself.

The immediate release phase begins supporting healthy glucose signaling and calming the digestive nervous system at bedtime before the first overnight metabolic disruption can occur, the control layer then holds all further release for approximately 1.5 to 2 hours allowing the body to settle into the early deep sleep cycles where growth hormone and insulin sensitivity are being reset before the delayed release phase activates precisely timed to the window when blood sugar instability most commonly bottoms out, cortisol surges in response, and the gut's overnight motility cycle either supports or sabotages the quality of sleep that remains.

TABLE 9
Sleep and metabolic support Formulation

| Components | amount |
|---|---|
| Immediate Release | |
| GABA | 10 mg-200 mg |
| L-Theanine | 20 mg-200 mg |
| Magnesium bisglycinate | 5 mg-250 mg |
| Melatonin | 0.5 mg-5 mg |
| Dihydroberberine (DHB) | 10 mg-125 mg |
| Cinnamon Extract | 15 mg-156 mg |
| Zinc bisglycinate | 20 mg-144 mg |
| Myo-Inositol | 25 mg-125 mg |
| Chromium | 20 mcg-200 mcg |
| Delayed Release | |
| Magnesium bisglycinate | 5 mg-250 mg |
| Melatonin | 0.5 mg-5 mg |
| Hops | 10 mg-100 mg |
| Lemon balm | 15 mg-125 mg |
| R-Alpha-Lipoic Acid (Na—R-ALA) | 10 mg-100 mg |
| Gymnema sylvestre extract | 10 mg-100 mg |
| Banaba leaf extract (2% corosolic | 3 mg-25 mg |
| acid) | |
| Ginger root extract | 5 mg-50 mg |
| Artichoke leaf extract | 15 mg-100 mg |

Composition for Sleep and GI Comfort (For Nighttime Nausea, Bloating, and the Digestive Slowdown That Keeps GLP-1 Users Awake). This is the first sleep supplement built specifically for the estimated 15 to 20 million Americans currently using GLP-1 medications and the millions more who will start in the coming years who are discovering that the same mechanism that suppresses appetite and slows gastric emptying during the day continues working overnight, creating a specific and predictable pattern of nighttime nausea, mid-sleep bloating, acid discomfort, and digestive restlessness that is not a side effect that goes away but a nightly biological reality that standard sleep aids are completely unequipped to address.

The immediate release phase calms the upper digestive nervous system and begins supporting gastric comfort at bedtime before the slowed gastric motility that GLP-1 activity produces can trigger the nausea and bloating that delays sleep onset, the control layer then holds all further release for approximately 1.5 to 2 hours, allowing the body to transition into sleep without digestive interruption before the delayed release phase activates precisely timed to the window when gastric emptying is at its most delayed, acid reflux risk peaks, and the discomfort that wakes GLP-1 users in the middle of the night is most likely to occur.

TABLE 10
Sleep and gastrointestinal comfort Formulation

| Components | amount |
|---|---|
| Immediate Release | |
| GABA | 10 mg-200 mg |
| L-Theanine | 20 mg-200 mg |
| Magnesium bisglycinate | 5 mg-250 mg |
| Melatonin | 0.5 mg-5 mg |
| Ginger root extract | 5 mg-130 mg |
| Gymnema sylvestre extract | 10 mg-125 mg |
| Akkermansia muciniphila | 5 mg-80 mg |
| Inulin | 3 mg-90 mg |
| Delayed Release | |
| Magnesium bisglycinate | 5 mg-250 mg |
| Melatonin | 0.5 mg-5 mg |
| Hops | 10 mg-100 mg |
| Lemon balm | 15 mg-125 mg |
| Berberine HCl | 25 mg-225 mg |
| Ceylon cinnamon bark extract | 10 mg-150 mg |
| Curcumin | 5 mg-150 mg |
| Bitter melon extract | 10 mg-130 mg |
| Trans-Resveratrol (from Polygonum | 10 mg-125 mg |
| cuspidatum) | |
| Fenugreek seed extract | 5 mg-150 mg |
| Chromium picolinate | 50 mcg-200 mcg |

Composition for Sleep and Digestion (Reduces Nighttime Heartburn, Acid Reflux, and Digestive Discomfort). This is the first sleep supplement built specifically for the estimated 60 million Americans who experience nighttime heartburn, acid reflux, or digestive discomfort at least once a week and who have learned through experience that lying down triggers the very symptoms that make falling asleep difficult, that the standard solution of propping up pillows addresses the position but never the underlying digestive chemistry, and that the only OTC products marketed for their condition are antacids and H2 blockers that treat the acid but leave the sleep entirely unaddressed.

The immediate release phase begins calming the upper digestive tract and supporting the mucosal lining that acid exposure compromises at bedtime, the control layer then holds all further release for approximately 1.5 to 2 hours allowing the body to move through the early sleep cycles before the stomach's acid production reaches its overnight peak before the Delayed Release phase activates precisely timed to the window between 1 and 3 AM when gastric acid secretion is at its highest, the lower esophageal sphincter is most relaxed in deep sleep, and the reflux events most likely to cause mid-night waking and morning throat discomfort are at their most disruptive.

TABLE 11
Sleep and digestion Formulation

| Components | amount |
|---|---|
| Immediate Release | |
| GABA | 10 mg-200 mg |
| L-Theanine | 20 mg-200 mg |
| Magnesium bisglycinate | 5 mg-250 mg |
| Melatonin | 0.5 mg-5 mg |
| Licorice root extract | 25 mg-312.5 mg |
| Slippery elm bark | 50 mg-250 mg |
| Ginger root extract | 25 mg-125 mg |
| Ashwagandha root extract | 25 mg-187.5 mg |
| Inulin | 3 mg-90 mg |
| Delayed Release | |
| Magnesium bisglycinate | 5 mg-250 mg |
| Melatonin | 0.5 mg-5 mg |
| Hops | 10 mg-100 mg |
| Lemon balm | 15 mg-125 mg |
| Zinc carnosine | 5 mg-125 mg |
| Licorice root extract | 37.5 mg-187.5 mg |
| L-Glutamine | 62.5 mg-312.5 mg |

### Hormonal Lifecycle Rhythm Support

Reproductive hormones including estrogen, progesterone, and testosterone, along with thyroid hormones and their downstream effects on thermoregulation and sleep architecture, follow both circadian and ultradian rhythms. Hormonal phase transitions such as perimenopause, menopause, and andropause disrupt these rhythms acutely, producing vasomotor symptoms, sleep fragmentation, and circadian phase instability.

Immediate release addresses sleep-onset difficulties associated with hormonal dysregulation including early-night vasomotor events. The control layer preserves the quiescent mid-sleep period. Delayed release supports the hormonal secretion windows active in the second half of the night, including testosterone pulsatility and progesterone-related sleep architecture maintenance.

Composition for Sleep and Thyroid Function (For disrupted sleep, temperature instability, and the metabolism that never quite resets overnight). This is the first sleep supplement built specifically for the estimated 20 million Americans living with thyroid conditions and the far larger population experiencing subclinical thyroid disruption, temperature dysregulation, and the metabolic sluggishness that never quite resolves no matter how many hours they spend in bed who have been told their thyroid levels are “normal” but continue to experience the racing heart at bedtime, the night sweats that soak through sheets, the 3 AM waking with a body that cannot decide if it is too hot or too cold, and the profound fatigue that greets them every morning regardless of how long they slept.

The immediate release phase begins supporting the adrenal and thyroid pathways that govern sleep-onset temperature regulation and calms the sympathetic nervous system hyperactivation that thyroid imbalance produces at bedtime, the control layer then holds all further release for approximately 1.5 to 2 hours allowing the body's core temperature to drop into the range that deep sleep requires without the thermoregulatory disruptions that thyroid dysfunction characteristically causes before the delayed release phase activates precisely timed to the window when thyroid hormone conversion from T4 to T3 is most active overnight, growth hormone secretion peaks, and the metabolic reset that thyroid-compromised sleepers are consistently denied is either completed or lost entirely depending on whether the second half of the night remains intact.

TABLE 12
Sleep and thyroid function Formulation

| Components | amount |
|---|---|
| Immediate Release | |
| GABA | 10 mg-200 mg |
| L-Theanine | 20 mg-200 mg |
| Magnesium bisglycinate | 5 mg-250 mg |
| Melatonin | 0.5 mg-5 mg |
| Ashwagandha root extract | 75 mg-375 mg |
| Selenium (selenium glycinate) | 50 mcg-250 mcg |
| Zinc bisglycinate | 4 mg-19 mg |
| L-Tyrosine | 62.5 mg-312.5 mg |
| Delayed Release | |
| Magnesium bisglycinate | 5 mg-250 mg |
| Melatonin | 0.5 mg-5 mg |
| Hops | 10 mg-100 mg |
| Lemon balm | 15 mg-125 mg |
| Guggul extract | 25 mg-125 mg |
| Schisandra berry extract | 25 mg-125 mg |
| Selenium (L-Selenomethionine) | 25 mcg-125 mcg |
| Zinc bisglycinate: | 2.5 mg-12.5 mg |

### Neural Recovery and Cognitive Restoration

Memory consolidation via hippocampal replay, glymphatic waste clearance, and synaptic homeostasis are sleep-stage-specific processes. Glymphatic clearance peaks in slow-wave sleep in the early part of the night. Memory consolidation concentrates in the REM-dominant second half. Both processes are temporally structured and disrupted by sleep fragmentation.

Immediate Release delivers neural calming and GABAergic support to facilitate entry into slow-wave sleep, when glymphatic clearance is most active. The control layer preserves this critical early deep-sleep window without interruption. Delayed release provides cognitive recovery and REM-supportive compounds timed to the second half of the night when REM sleep is predominant.

Memory consolidation and glymphatic clearance are among the most time-specific biological processes in the overnight sleep cycle. The formulation delivers neural calming for the early SWS glymphatic window and cognitive repair compounds for the REM-dominant second half of the night.

Composition for Sleep and Cognitive Recovery (For the blue light hangover, the wired-but-tired feeling at bedtime, and the brain fog that screens leave behind). This is the first sleep supplement built specifically for the estimated 200 million Americans who spend six or more hours a day on screens and have experienced firsthand the paradox that defines modern exhaustion the body is physically tired, the eyes are burning, the brain is foggy, but the moment the screen goes dark the mind accelerates, cortisol is elevated, melatonin is suppressed, and the nervous system is running at a frequency that makes genuine restorative sleep feel like something that happens to other people a condition that no amount of blue light glasses, screen-free wind-down time, or standard melatonin addresses because the damage to sleep architecture from sustained screen exposure is neurochemical, not behavioral, and requires a targeted overnight recovery response that begins at bedtime and continues through the middle of the night.

The immediate release phase begins counteracting the melatonin suppression, cortisol elevation, and dopaminergic overstimulation that screen exposure produces at bedtime, calming the wired-but-tired nervous system and beginning the neurochemical reset that makes sleep onset possible. The Control Layer then holds all further release for approximately 1.5 to 2 hours. Allowing the brain to transition fully into the slow-wave deep sleep where the neural recovery from sustained cognitive stimulation actually occurs before the delayed release phase activates precisely timed to the REM-dominant second half of the night when the brain consolidates the day's information, clears the cognitive residue of hours of digital stimulation, and either completes or fails to complete the overnight restoration that determines whether tomorrow begins with clarity or fog.

TABLE 13
Sleep and cognitive recovery Formulation

| Components | amount |
|---|---|
| Immediate Release | |
| GABA | 10 mg-200 mg |
| L-Theanine | 20 mg-200 mg |
| Magnesium bisglycinate | 5 mg-250 mg |
| Melatonin | 0.5 mg-5 mg |
| Phosphatidylserine | 25 mg-125 mg |
| Ashwagandha root extract | 25 mg-250 mg |
| Lutein | 2 mg-25 mg |
| Delayed Release | |
| Magnesium bisglycinate | 5 mg-250 mg |
| Melatonin | 0.5 mg-5 mg |
| Hops | 10 mg-100 mg |
| Lemon balm | 15 mg-125 mg |
| Lion's mane extract | 125 mg-625 mg |
| CDP-Choline (Citicoline) | 37.5 mg-187.5 mg |
| Bacopa monnieri extract | 37.5 mg-187.5 mg |

### Immune and Cellular Repair Rhythms

Immune surveillance, cytokine release, and cellular repair processes including growth hormone pulsatility and tissue regeneration are concentrated in the early slow-wave sleep window. NK cell activity, antibody synthesis, and anti-inflammatory cytokine release are sleep-dependent and time-gated. Sleep deprivation acutely suppresses these functions.

Immediate Release provides initial immune-supportive ingredients at ingestion. The Control Layer maintains a quiescent period allowing transition into deep sleep, when immune activity peaks. Delayed Release delivers immune-modulating and cellular repair compounds timed to the deep sleep window approximately 1.5 to 2 hours after ingestion, at the precise biological moment when these processes are most active.

Composition for Sleep and Physical Recovery (For athletes, post-illness recovery, and chronic fatigue). This is the first sleep supplement built specifically for the estimated 60 million Americans who push their bodies beyond what ordinary rest can repair. Competitive athletes and weekend warriors whose muscles are still breaking down hours after training ends. The 18 million living with post-illness fatigue and long-term recovery challenges, and the tens of millions experiencing the chronic exhaustion that accumulates when physical demand consistently outpaces the body's ability to restore itself overnight, all of whom share a single biological reality that no standard sleep aid addresses. The restorative processes the body needs most: growth hormone secretion, muscle protein synthesis, immune reconstitution, and cellular repair unfold in a precise overnight sequence that disrupted or insufficient sleep interrupts before it can complete.

The immediate release phase begins supporting the hormonal and immune conditions for deep physical restoration at bedtime calming systemic inflammation, preparing the neuroendocrine environment for peak growth hormone release, and beginning the cellular repair signaling that the first deep sleep cycle then accelerates. The control layer then holds all further release for approximately 1.5 to 2 hours protecting the critical slow-wave sleep window where growth hormone reaches its highest overnight concentration and where the majority of physical tissue repair is initiated before the delayed release phase activates. The delayed release phase actives precisely timed to the second half of the night when immune reconstitution is most active, oxidative stress from intense physical exertion peaks in muscle tissue, and the anabolic and anti-inflammatory processes that separate genuine recovery from mere rest are either completed or abandoned depending on whether the sleep architecture remains intact.

TABLE 14
Sleep and physical recovery Formulation

| Components | amount |
|---|---|
| Immediate Release | |
| GABA | 10 mg-200 mg |
| L-Theanine | 20 mg-200 mg |
| Magnesium bisglycinate | 5 mg-250 mg |
| Melatonin | 0.5 mg-5 mg |
| Ashwagandha root extract | 50 mg-250 mg |
| Vitamin C | 62.5 mg-312.5 mg |
| Zinc bisglycinate | 25 mg-144 mg |
| Delayed Release | |
| Magnesium bisglycinate | 5 mg-250 mg |
| Melatonin | 0.5 mg-5 mg |
| Hops | 10 mg-100 mg |
| Lemon balm | 15 mg-125 mg |
| Reishi mushroom extract | 125 mg-625 mg |
| Astragalus root extract | 62.5 mg-312.5 mg |

### Additional Exemplary Formulation

Composition for Sleep and Physical Comfort is the first sleep supplement designed specifically for the estimated 50 million Americans whose physical discomfort keeps them from falling asleep or wakes them in the middle of the night delivering a precisely timed three-phase response that no single-release melatonin or antihistamine product can replicate.

The immediate release phase quiets the nervous system and begins calming physical tension at bedtime, the control layer then holds all further release for approximately 1.5 to 2 hours. Thus, allowing the body to transition fully into sleep undisturbed before the delayed release phase activates precisely timed to the overnight window when the body's own anti-inflammatory defenses drop to their lowest point and physical discomfort most commonly intensifies.

TABLE 15
Sleep and physical comfort Formulation

| Components | amount |
|---|---|
| Immediate Release | |
| GABA | 10 mg-200 mg |
| L-Theanine | 20 mg-200 mg |
| Magnesium bisglycinate | 5 mg-250 mg |
| Melatonin | 0.5 mg-5 mg |
| Palmitoylethanolamide | 75 mg-375 mg |
| Tart cherry extract | 120 mg-600 mg |
| Stem bromelain, (2,400 GDU/g) | 62.5 mg-312.5 mg |
| Delayed Release | |
| Magnesium bisglycinate | 5 mg-250 mg |
| Melatonin | 0.5 mg-5 mg |
| Hops | 10 mg-100 mg |
| Lemon balm | 15 mg-125 mg |
| Boswellia serrata extract: | 25 mg-125 mg |
| Collagen (25% undenatured type | 10 mg-50 mg |
| II collagen): | |
| Vitamin D3 | 500 IU-2,500 IU |

Composition for Sleep and Leg Comfort is the first sleep supplement built specifically for the estimated 25 to 35 million Americans whose legs won't let them sleep delivering a precisely timed three-phase response that targets the sensory discomfort that prevents sleep onset and the involuntary leg movements that fragment sleep throughout the night.

The immediate release phase calms the nervous system and supports the dopaminergic and iron-related pathways that drive the uncomfortable crawling and tingling sensations at bedtime. The control layer then holds all further release for approximately 1.5 to 2 hours allowing the body to transition into sleep without interruption before the delayed release phase activates precisely timed to the deep sleep window when periodic limb movements are most disruptive and most likely to cause mid-night waking.

TABLE 16
Sleep and leg comfort Formulation

| Components | amount |
|---|---|
| Immediate Release | |
| GABA | 10 mg-200 mg |
| L-Theanine | 20 mg-200 mg |
| Magnesium bisglycinate | 5 mg-250 mg |
| Melatonin | 0.5 mg-5 mg |
| Iron bisglycinate | 4.5 mg-22.5 mg |
| Vitamin B6 | 10 mg-50 mg |
| Vitamin C | 25 mg-125 mg |
| Delayed Release | |
| Magnesium bisglycinate | 5 mg-250 mg |
| Melatonin | 0.5 mg-5 mg |
| Hops | 10 mg-100 mg |
| Lemon balm | 15 mg-125 mg |
| Folate methylfolate | 100 mcg-500 mcg |
| Vitamin E mixed tocopherols (d- | 50 IU-250 IU |
| alpha + gamma) | |
| Zinc bisglycinate | 2.5 mg-12.5 mg |

Composition for Sleep and Neurotransmitter Recovery is the first sleep supplement built specifically for the estimated 70 million Americans who drink alcohol regularly and know firsthand that even a few drinks at night produces the kind of fragmented, restless, unrestorative sleep that leaves them exhausted the next day delivering a precisely timed three-phase response engineered to match the exact two-stage way alcohol disrupts sleep architecture.

The immediate release phase calms the nervous system and begins supporting the GABA pathways that alcohol initially sedates at bedtime. The control layer then holds all further release for approximately 1.5 to 2 hours allowing the body's natural sleep onset to proceed undisturbed before the delayed release phase activates precisely timed to the window when alcohol's sedative effect wears off, GABA rebound begins, and the body shifts into the fragmented, cortisol-spiked second half of the night that after-hours sleepers know all too well.

TABLE 17
Sleep and neurotransmitter recovery Formulation

| Components | amount |
|---|---|
| Immediate Release | |
| GABA | 10 mg-200 mg |
| L-Theanine | 20 mg-200 mg |
| Magnesium bisglycinate | 5 mg-250 mg |
| Melatonin | 0.5 mg-5 mg |
| Dihydromyricetin (DHM) | 75 mg-375 mg |
| Hovenia dulcis extract | |
| Milk thistle silymarin extract | 37.5 mg-187.5 mg |
| Vitamin B or Benfotiamine: | 12.5 mg-62.5 mg |
| Delayed Release | |
| Magnesium bisglycinate | 5 mg-250 mg |
| Melatonin | 0.5 mg-5 mg |
| Hops | 10 mg-100 mg |
| Lemon balm | 15 mg-125 mg |
| Vitamin B6 | 6.25 mg-31.25 mg |
| Vitamin C or calcium ascorbate | 62.5 mg-312.5 mg |
| Zinc bisglycinate | 2.5 mg-12.5 mg |

Composition for Sleep and Joint is the first sleep supplement built specifically for the estimated 58 million Americans living with arthritis and joint discomfort who understand that the relationship between joint stiffness and sleep is not one-directional joint discomfort disrupts sleep, and poor sleep makes joint discomfort worse the next day delivering a precisely timed three-phase response that breaks that cycle at both ends of the night.

The immediate release phase calms the nervous system and begins easing joint tension and the physical restlessness that makes it difficult to fall asleep, the Control Layer then holds all further release for approximately 1.5 to 2 hours allowing the body to settle fully into the early deep sleep cycles where physical restoration begins before the Delayed Release phase activates precisely timed to the overnight window when pro-inflammatory cytokines reach their peak, morning stiffness is being set in motion, and the body's own anti-inflammatory defenses are at their lowest point.

TABLE 18
Sleep and joint Formulation

| Components | amount |
|---|---|
| Immediate Release | |
| GABA | 10 mg-200 mg |
| L-Theanine | 20 mg-200 mg |
| Magnesium bisglycinate | 5 mg-250 mg |
| Melatonin | 0.5 mg-5 mg |
| Glucosamine sulfate | 187.5 mg-937.5 mg |
| Curcumin | 62.5 mg-312.5 mg |
| Palmitoylethanolamide | 75 mg-375 mg |
| Delayed Release | |
| Magnesium bisglycinate | 5 mg-250 mg |
| Melatonin | 0.5 mg-5 mg |
| Hops | 10 mg-100 mg |
| Lemon balm | 15 mg-125 mg |
| Boswellia serrata | 25 mg-125 mg |
| Collagen (25% undenatured type | 10 mg-50 mg |
| II collagen) | |
| Chondroitin sulfate | 50 mg-250 mg |

Composition for Sleep and Neuroprotection This is the first sleep supplement built specifically for the estimated 115 million Americans over 40 who wake up foggy, forgetful, and mentally fatigued- and who are beginning to understand that the brain's overnight restoration process, including the glymphatic system's clearance of metabolic waste that accumulates during the day, depends entirely on the quality and architecture of the sleep they get each night-delivering a precisely timed three-phase response engineered to protect and extend the deep sleep cycles where that restoration is most active.

The Immediate Release phase calms the nervous system and supports the neurochemical conditions for deep sleep onset at bedtime, the Control Layer then holds all further release for approximately 1.5 to 2 hours—protecting the brain's critical early slow-wave sleep cycles from disruption—before the Delayed Release phase activates precisely timed to the second half of the night when REM sleep dominates, memory consolidation is most active, and neuroprotective processes require sustained nutritional support to complete their work. 10

TABLE 19
Sleep and neuroprotection Formulation

| Components | amount |
|---|---|
| Immediate Release | |
| GABA | 10 mg-200 mg |
| L-Theanine | 20 mg-200 mg |
| Magnesium bisglycinate | 5 mg-250 mg |
| Melatonin | 0.5 mg-5 mg |
| Lion's Mane Hericium Erinaceus | 125 mg-625 mg |
| fruiting body extract | |
| Phosphatidylserine (sunflower- | 25 mg-125 mg |
| derived, soy-free) | |
| Ashwagandha root extract | 50 mg-250 mg |
| Delayed Release | |
| Magnesium bisglycinate | 5 mg-250 mg |
| Melatonin | 0.5 mg-5 mg |
| Hops | 10 mg-100 mg |
| Lemon balm | 15 mg-125 mg |
| CDP-Choline (Citicoline) | 62.5 mg-312.5 mg |
| Bacopa Monnieri | 37.5 mg-187.5 mg |
| Pterostilbenep | 12.5 mg-62.5 mg |

### Exemplary Embodiments

Embodiment 1: A delivery capsule comprising a first population of lipid-based beads and a second population of lipid-based beads;
wherein the first population of lipid-based beads encapsulates one or more sleep promoting active agents; and the second population of beads encapsulates one or more sleep quality active agents;
wherein after administration of the delivery capsule the first population of lipid-based beads has an immediate burst release of the sleep promoting active agents, and the second population of lipid-based beads has a sustained release of the sleep quality active agents; and
wherein each active agent is independently a phytochemical, a herbal extract, a vitamin, a hormone, an amino acid, a mineral, enzymes, neurotransmitters, or combinations thereof.

Embodiment 2: The delivery capsule of embodiment 1, wherein the first population of lipid-based beads, second population of lipid-based beads, or any combination thereof further comprises one or more support active agent (e.g., In some embodiments, the support active agents can include, but are not limited to, one or more sleep support active agents, one or more menopause support active agents, one or more prostate support active agents, one or more urinary support active agents, pain relief active agent, restless leg relief active agent, gastrointestinal health active agent, hormonal support active agent (e.g., thyroid function), digestive support active agent, weight management active agent, immune support active agent, energy renewal active agent, physical recovery active agent, neurotransmitter recovery active agent, neuroprotection support active agent, stress relief active agent, mood balance active agent, skin and hair support active agent, jet lag relief active agent, muscle relaxation active agent, bone/joint support active agent, cognitive support active agent, respiratory support active agent, metabolic support (e.g., blood sugar balance) active agent, heart support active agent, or any combination thereof.)
one or more additional active agents, or any combination thereof.

Embodiment 3: The delivery capsule of any one of embodiments 1-2, wherein the first population of lipid-based beads further comprises one or more support active agent.

Embodiment 4: The delivery capsule of any one of embodiments 1-3, wherein the first population of lipid-based beads has an immediate burst release of the sleep promoting active agent within about 30 minutes of administration of the capsule.

Embodiment 5: The delivery capsule of any one of embodiments 1-4, wherein at least 80% (e.g., at least 90%, at least 91%, at least 92.5%, at least 95%, at least 97.5%, or at least 99%) of the sleep promoting active agents are released from the first population of beads within 2 hours of administration of the capsule.

Embodiment 6: The delivery capsule of any one of embodiments 1-5, wherein the second population of lipid-based beads has a sustained release of the sleep quality active agent from about 10 minutes to about 8 hours after administration of the capsule.

Embodiment 7: The delivery capsule of any one of embodiments 1-6, wherein there is no active agent release between the immediate burst release of the sleep promoting active agent and the sustained release of the sleep quality agent.

Embodiment 8: The delivery capsule of any one of embodiments 1-7, wherein sustained release of the sleep quality active agents is selected from a delayed burst release, a delayed sustained release, and combinations thereof.

Embodiment 9: The delivery capsule of any one of embodiments 1-8, wherein sustained release of the sleep quality active agents comprises a delayed sustained release of the sleep quality active agents.

Embodiment 10: The delivery capsule of any one of embodiments 1-9, wherein delayed sustained release of at least a portion of the sleep quality active agents is within 2 to 5 hours of administration.

Embodiment 11: The delivery capsule of any one of embodiments 1-10, wherein the sleep quality active agent has a delayed sustained release of 2.5-4.5 hours.

Embodiment 12: The delivery capsule of any one of embodiments 1-11, wherein the first population of lipid-based beads comprises a wax.

Embodiment 13: The delivery capsule of any one of embodiments 1-12, wherein the second population of lipid-based beads comprises a wax.

Embodiment 14: The delivery capsule of embodiment 13, wherein the wax is glycerol monosterate, carnauba wax, yellow wax, white beeswax, cetyl palmitate, rice bran wax, candelilla wax, hydrogenated vegetable oils, stearic acid, stearyl alcohol, microcrystalline cellulose wax blends, polyethylene glycol waxes, montan wax, or any combination thereof.

Embodiment 15: The delivery capsule of any one of embodiments 12-14, wherein the wax is present in a concentration of from 20% by weight to 80% by weight (e.g., from 40% to 60% by weight) based on a total weight of the first population of lipid-based beads.

Embodiment 16: The delivery capsule of any one of embodiments 12-15, wherein the wax is present in a concentration of from 20% by weight to 80% by weight (e.g., from 40% to 60% by weight) based on a total weight of the second population of lipid-based beads.

Embodiment 17: The delivery capsule of any one of embodiments 1-16, wherein the second population of lipid-based beads comprises a binder.

Embodiment 18: The delivery capsule of embodiment 17, wherein the binder comprises methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC), hydroxyethylcarboxymethylcellulose (HECMC), carboxymethylhydroxyethylcellulose (CMHEC), or pharmaceutically acceptable salts thereof, or any combination thereof.

Embodiment 19: The delivery capsule of any one of embodiments 17-18, wherein the second population of lipid-based beads comprises hydroxypropyl methyl cellulose (HPMC).

Embodiment 20: The delivery capsule of any one of embodiments 17-19, wherein the binder is present in a concentration of from 0.1% by weight to 20% by weight (e.g., from 0.5% by weight to 1.5% by weight) based on a total weight of the second population of lipid-based beads.

Embodiment 21: The delivery capsule of any one of embodiments 17-20, wherein the wax and the binder are present in the second population of lipid-based beads in a ratio of wax to binder of from 5:1 to 150:1 (e.g., from 110:1 to 140:1).

Embodiment 22: The delivery capsule of any one of embodiments 1-21, wherein the sleep promoting active agent comprises melatonin, L-theanine, γ-aminobutyric acid (GABA), magnesium, or any combination thereof.

Embodiment 23: The delivery capsule of any one of embodiments 1-22, wherein the first population of lipid-based beads comprises at least two sleep promoting active agents selected from the group consisting of melatonin, L-theanine, γ-aminobutyric acid (GABA), and magnesium.

Embodiment 24: The delivery capsule of any one of embodiments 1-23, wherein the one or more sleep promoting active agents are present in a concentration of from 5% by weight to 80% by weight (e.g., from 40% by weight to 60% by weight) based on a total weight of the first population of lipid based beads.

Embodiment 25: The delivery capsule of any one of embodiments 2-24, wherein the first population of lipid-based beads, second population of lipid-based beads, or any combination thereof comprises a menopause support active agent.

Embodiment 26: The delivery capsule of any one of embodiments 2-25, wherein the menopause support active agent comprises red cover, ashwagandha extract, folic acid, vitamin D, or any combination thereof.

Embodiment 27: The delivery capsule of any one of embodiments 2-26, wherein the first population of lipid-based beads, second population of lipid-based beads, or any combination thereof comprises a urinary support active agent.

Embodiment 28: The delivery capsule of any one of embodiments 2-24, and 27, wherein the urinary support active agent comprises β-sitosterol, saw palmetto, nettle extract, vitamin D, selenium, or any combination thereof.

Embodiment 29: The delivery capsule of any one of embodiments 1-28, wherein the sleep quality active agent comprises hops extract, lemon balm extract, valerian root extract, melatonin, or any combination thereof.

Embodiment 30: The delivery capsule of any one of embodiments 1-29, wherein the second population of lipid-based beads comprises at least two sleep quality active agents selected from the group consisting of hops extract, lemon balm extract, valerian root extract, and melatonin.

Embodiment 31: The delivery capsule of any one of embodiments 1-30, wherein the second population of lipid-based beads comprises hops extract, lemon balm extract, and melatonin.

Embodiment 32: The delivery capsule of any one of embodiments 1-31, wherein the one or more sleep quality active agents are present in a concentration of from 5% by weight to 80% by weight (e.g., from 40% by weight to 60% by weight) based on a total weight of the second population of lipid based beads.

Embodiment 33: The delivery capsule of any one of embodiments 22-32, wherein when present the melatonin is present in a concentration of from 0.01% by weight to 30% by weight (e.g., from 0.5% by weight to 1.5% by weight) based on a total weight of the first population of lipid-based beads.

Embodiment 34: The delivery capsule of any one of embodiments 22-33, wherein when present the melatonin is present in a concentration of from 0.01% by weight to 30% by weight (e.g., from 0.25% by weight to 1% by weight) based on a total weight of the second population of lipid-based beads.

Embodiment 35: The delivery capsule of any one of embodiments 22-34, wherein when present the melatonin is present in a concentration of from 0.005% by weight to 5% by weight (e.g., from 0.25% by weight to 2% by weight) based on a total weight of the delivery capsule.

Embodiment 36: The delivery capsule of any one of embodiments 1-35, wherein the first population of lipid-based beads and the second population of lipid-based beads are present in the delivery capsule in a ratio of first population of lipid-based beads to second population of lipid-based beads of from 1:1 to 1:10 (e.g., from 1:1 to 1:2).

Embodiment 37: The delivery capsule of any one of embodiments 1-36, wherein capsule comprises the components in proportions as set forth in Tables 1-19.

Embodiment 38: A method for treating a sleep disorder or modifying or improving a sleep-wake cycle in a subject comprising, administering:
a delivery capsule comprising a first population of lipid-based beads and a second population of lipid-based beads;
wherein the first population of lipid-based beads encapsulates one or more sleep promoting active agents; and the second population of beads encapsulates one or more sleep quality active agents;
wherein after administration of the delivery capsule the first population of lipid-based beads has an immediate burst release of the sleep promoting active agents, and the second population of lipid-based beads has a sustained release of the sleep quality active agents; and
wherein each active agent is independently a phytochemical, a herbal extract, a vitamin, a hormone, an amino acid, a mineral, enzymes, neurotransmitters, or combinations thereof.

Embodiment 39: The method of embodiment 38, wherein the first population of lipid-based beads, second population of lipid-based beads, or any combination thereof further comprises one or more support active agent (e.g., one or more sleep support active agents, one or more menopause support active agents, one or more prostate support active agents, one or more bladder support active agents, one or more urinary support active agents, or any combination thereof), one or more additional active agents, or any combination thereof.

Embodiment 40: The method of any one of embodiments 38-39, comprising: administering a delivery capsule according to any one of claims 1-37.

Embodiment 41: The method of any one of embodiments 38-40, wherein the sleep disorder includes insomnia, narcolepsy, sleep apnea, hypersomnia, and combinations thereof.

Embodiment 42: The method of any one of embodiments 38-41, wherein the subject is a menopausal subject.

Embodiment 43: The method of embodiment 42, wherein the menopausal subject has at least one of the following symptoms sweating secondary to vasomotor instability, night sweats, hot flashes fatigue, irritability, insomnia, inability to concentrate, depression, memory loss, headache, anxiety, nervousness, intermittent dizziness, paresthesias, palpitations and tachycardia as well as nausea, constipation, diarrhea, arthralgia, myalgia, cold hands and feet, weight gain, changes to the genitals, urinary incontinence, vaginal dryness, loss of pelvic muscle tone, increased risk of cardiovascular disease, or osteoporosis.

Embodiment 44: A method of reducing a symptom of menopause, the method comprising administering to a menopausal subject a delivery capsule comprising a first population of lipid-based beads and a second population of lipid-based beads;
wherein the first population of lipid-based beads encapsulates one or more sleep promoting active agents and one or more menopause support active agents; and the second population of beads encapsulates one or more sleep quality active agents;
wherein after administration of the delivery capsule the first population of lipid-based beads has an immediate burst release of the sleep promoting active agents and one or more menopause support active agents, and the second population of lipid-based beads has a sustained release of the sleep quality active agents; and
wherein each active agent is independently a phytochemical, a herbal extract, a vitamin, a hormone, an amino acid, a mineral, enzymes, neurotransmitters, or combinations thereof.

Embodiment 45: The method of embodiment 44, comprising: administering a delivery capsule according to any one of embodiments 1-37.

Embodiment 46: The method of embodiment 44 or embodiment 45, wherein the symptom is sweating secondary to vasomotor instability, night sweats, hot flashes, fatigue, irritability, insomnia, anxiety, or any combination thereof.

Embodiment 47: A method for treating a sleep disorder or modifying or improving a sleep-wake cycle and reducing a symptom of menopause in a menopausal subject, the method comprising: administering to a menopausal subject a delivery capsule comprising a first population of lipid-based beads and a second population of lipid-based beads;
wherein the first population of lipid-based beads encapsulates one or more sleep promoting active agents and one or more menopause support active agents; and the second population of beads encapsulates one or more sleep quality active agents;
wherein after administration of the delivery capsule the first population of lipid-based beads has an immediate burst release of the sleep promoting active agents and one or more menopause support active agents, and the second population of lipid-based beads has a sustained release of the sleep quality active agents; and
wherein each active agent is independently a phytochemical, a herbal extract, a vitamin, a hormone, an amino acid, a mineral, enzymes, neurotransmitters, or combinations thereof.

Embodiment 48: The method of embodiment 47, comprising: administering a delivery capsule according to any one of claims 1-37.

Embodiment 49: The method of any one of embodiments 47-48, wherein the sleep disorder includes insomnia, narcolepsy, sleep apnea, hypersomnia, and combinations thereof.

Embodiment 50: The method of any one of embodiments 47-49, wherein the symptom is sweating, secondary to vasomotor instability, night sweats, hot flashes, fatigue, irritability, insomnia, anxiety, or any combination thereof.

Embodiment 51: A method for reducing urinary frequency during sleep-wake cycle in a subject, the method comprising: administering to the subject a delivery capsule comprising a first population of lipid-based beads and a second population of lipid-based beads;
wherein the first population of lipid-based beads encapsulates one or more sleep promoting active agents and one or more urinary support active agents; and the second population of beads encapsulates one or more sleep quality active agents;
wherein after administration of the delivery capsule the first population of lipid-based beads has an immediate burst release of the sleep promoting active agents and one or more urinary support active agents, and the second population of lipid-based beads has a sustained release of the sleep quality active agents; and
wherein each active agent is independently a phytochemical, a herbal extract, a vitamin, a hormone, an amino acid, a mineral, enzymes, neurotransmitters, or combinations thereof.

Embodiment 52: The method of embodiment 51, comprising: administering a delivery capsule according to any one of claims 1-37.

Embodiment 53: A method for reducing urinary frequency during sleep-wake cycle and treating a sleep disorder or modifying or improving the sleep-wake cycle in a subject, the method comprising: administering to the subject a delivery capsule comprising a first population of lipid-based beads and a second population of lipid-based beads;
wherein the first population of lipid-based beads encapsulates one or more sleep promoting active agents and one or more urinary support active agents; and the second population of beads encapsulates one or more sleep quality active agents;
wherein after administration of the delivery capsule the first population of lipid-based beads has an immediate burst release of the sleep promoting active agents and one or more urinary support active agents, and the second population of lipid-based beads has a sustained release of the sleep quality active agents; and
wherein each active agent is independently a phytochemical, a herbal extract, a vitamin, a hormone, an amino acid, a mineral, enzymes, neurotransmitters, or combinations thereof.

Embodiment 54: The method of embodiment 53, comprising: administering a delivery capsule according to any one of claims 1-37.

Embodiment 55: The method of any one of embodiments 53-54, wherein the sleep disorder includes insomnia, narcolepsy, sleep apnea, hypersomnia, and combinations thereof.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

What is claimed is:

1. A delivery capsule comprising a first population of lipid-based beads and a second population of lipid-based beads;
wherein the first population of lipid-based beads encapsulates one or more sleep promoting active agents; and the second population of beads encapsulates one or more sleep quality active agents;
wherein after administration of the delivery capsule the first population of lipid-based beads has an immediate burst release of the sleep promoting active agents, and the second population of lipid-based beads has a sustained release of the sleep quality active agents; and
wherein each active agent is independently a phytochemical, a herbal extract, a vitamin, a hormone, an amino acid, a mineral, enzymes, neurotransmitters, or combinations thereof.

2. The delivery capsule of claim 1, wherein the first population of lipid-based beads, second population of lipid-based beads, or any combination thereof further comprises one or more support active agent, one or more additional active agents, or any combination thereof.

3. The delivery capsule of claim 1, wherein the first population of lipid-based beads has an immediate burst release of the sleep promoting active agent within about 30 minutes of administration of the capsule.

4. The delivery capsule of claim 1, wherein at least 80% of the sleep promoting active agents are released from the first population of beads within 2 hours of administration of the capsule.

5. The delivery capsule of claim 1, wherein the second population of lipid-based beads has a sustained release of the sleep quality active agent from about 10 minutes to about 8 hours after administration of the capsule.

6. The delivery capsule of claim 1, wherein there is no active agent release between the immediate burst release of the sleep promoting active agent and the sustained release of the sleep quality agent.

7. The delivery capsule of claim 1, wherein sustained release of the sleep quality active agents comprises a delayed sustained release of the sleep quality active agents.

8. The delivery capsule of claim 1, wherein the delayed sustained release of at least a portion of the sleep quality active agents is within 2 to 5 hours of administration.

9. The delivery capsule of claim 1, wherein the first population of lipid-based beads, the second population of lipid-based beads, or any combination thereof comprises a wax.

10. The delivery capsule of claim 1, wherein the second population of lipid-based beads comprises a binder.

11. The delivery capsule of claim 10, wherein the binder comprises methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC), hydroxyethylcarboxymethylcellulose (HECMC), carboxymethylhydroxyethylcellulose (CMHEC), or pharmaceutically acceptable salts thereof, or any combination thereof.

12. The delivery capsule of claim 10, wherein the wax and the binder are present in the second population of lipid-based beads in a ratio of wax to binder of from 5:1 to 150:1.

13. The delivery capsule of claim 1, wherein the sleep promoting active agent comprises melatonin, L-theanine, γ-aminobutyric acid (GABA), magnesium, or any combination thereof.

14. The delivery capsule of claim 2, wherein when the support active agent is present in the first population of lipid-based beads, second population of lipid-based beads, or any combination thereof, the support active agent comprises a menopause support active agent, or a urinary support active agent.

15. The delivery capsule of claim 2, wherein
when the menopause support active agent is present the menopause support active agent comprises red cover, ashwagandha extract, folic acid, vitamin D, or any combination thereof, or
when the urinary support active agent is present the urinary support active agent comprises β-sitosterol, saw palmetto, nettle extract, vitamin D, selenium, or any combination thereof.

16. The delivery capsule of claim 1, wherein the sleep quality active agent comprises hops extract, lemon balm extract, valerian root extract, melatonin, or any combination thereof.

17. The delivery capsule of claim 1, wherein the first population of lipid-based beads and the second population of lipid-based beads are present in the delivery capsule in a ratio of first population of lipid-based beads to second population of lipid-based beads of from 1:1 to 1:10.

18. A method for treating a sleep disorder or modifying or improving a sleep-wake cycle in a subject comprising, administering a delivery capsule of claim 1.

19. A method of reducing a symptom of menopause in a subject or for reducing urinary frequency during sleep-wake cycle in a subject, the method comprising administering to a subject a delivery capsule comprising a first population of lipid-based beads and a second population of lipid-based beads;
wherein the first population of lipid-based beads encapsulates one or more sleep promoting active agents and one or more menopause support active agents or one or more urinary support active agents; and the second population of beads encapsulates one or more sleep quality active agents;
wherein after administration of the delivery capsule the first population of lipid-based beads has an immediate burst release of the sleep promoting active agents and one or more menopause support active agents or one or more urinary support active agents, and the second population of lipid-based beads has a sustained release of the sleep quality active agents; and
wherein each active agent is independently a phytochemical, a herbal extract, a vitamin, a hormone, an amino acid, a mineral, enzymes, neurotransmitters, or combinations thereof.

20. A method for treating a sleep disorder or modifying or improving a sleep-wake cycle and reducing a symptom of menopause in a subject or reducing urinary frequency during sleep-wake cycle in a subject, the method comprising:
administering to the subject a delivery capsule comprising a first population of lipid-based beads and a second population of lipid-based beads;
wherein the first population of lipid-based beads encapsulates one or more sleep promoting active agents and one or more menopause support active agents or one or more urinary support active agents; and the second population of beads encapsulates one or more sleep quality active agents;
wherein after administration of the delivery capsule the first population of lipid-based beads has an immediate burst release of the sleep promoting active agents and one or more menopause support active agents or one or more urinary support active agents, and the second population of lipid-based beads has a sustained release of the sleep quality active agents; and
wherein each active agent is independently a phytochemical, a herbal extract, a vitamin, a hormone, an amino acid, a mineral, enzymes, neurotransmitters, or combinations thereof.
